(19) United States
(12) Patent Application Publication
(10) Pub. No.: US 2024/0189230 A1
(43) Pub. Date: Jun. 13, 2024

(54) PALM FOR THE TREATMENT OF CHEMOTHERAPY-INDUCED PERIPHERAL NEUROPATHY INCIDENTAL TO THE TREATMENT OF CANCER

(71) Applicant: Peptinovo Biopharma Inc., Ann Arbor, MI (US)

(72) Inventor: Reynold Homan, Ann Arbor, MI (US)

(21) Appl. No.: 17/634,975

(22) PCT Filed: Aug. 11, 2020
(86) PCT No.: PCT/US2020/045785

§ 371 Date: Feb. 12, 2022

Related U.S. Application Data
(60) Provisional application No. 62/886,282, filed on Aug. 13, 2019.

(51) Int. Cl. A61K 9/107; A61K 31/167; A61K 31/282; A61K 31/337; A61K 31/4745; A61K 33/243; A61K 38/10; A61K 38/16; A61K 47/24; A61K 47/28; A61K 47/54; A61K 47/69; A61K 49/18; A61K 51/12; A61P 35/00

(52) U.S. Cl. CPC A61K 9/1075; A61K 31/167; A61K 31/282; A61K 31/337; A61K 31/4745; A61K 33/243; A61K 38/10; A61K 38/16; A61K 47/24; A61K 47/28; A61K 47/545; A61K 47/6909; A61K 49/1809; A61K 51/1227; A61P 35/00

(57) ABSTRACT

The present disclosure provides a method for the treatment or prevention of Chemotherapy-Induced Peripheral Neuropathy (CIPN) in a cancer patient treated with, or to be treated with, a CIPN causing chemotherapeutic agent, the method comprising: administering a therapeutically effective amount of a composition containing a peptide amphiphile lipid micelle (PALM) nanoparticle to the cancer patient, the PALM nanoparticle comprising a PALM containing the CIPN causing chemotherapeutic agent, and wherein the PALM comprises a peptide, and a lipid component comprising sphingomyelin and one or more additional phospholipids.

### CROSS REFERENCE TO RELATED APPLICATIONS

This application is a 371 of International Application of PCT/US2020/045785, filed on Aug. 11, 2020, which claims the benefit of U.S. Provisional Application Ser. No. 62/886,282, filed Aug. 13, 2019. The entire contents of the aforementioned disclosures are hereby incorporated by reference.

### SEQUENCE LISTING

This application incorporates by reference in its entirety the sequence listing entitled “236603_471132_SequenceListing_ST25.txt”, (30KB) which was created on Feb. 7, 2022, and filed electronically herewith.

### FIELD

The present invention relates to the treatment of cancer using peptide amphiphile lipid micelles (PALM), wherein such use prevents, treats, ameliorates or diminishes adverse effects, including chemotherapy-induced peripheral neuropathy (CIPN) caused by the administration of a chemotherapeutic agent. More particularly, the present invention concerns a formulation technology enabling the incorporation of CIPN inducing chemotherapy drugs into nanoparticles that can be readily administered parenterally for the safe and effective delivery of the incorporated chemotherapy drugs to their therapeutic targets and diminishes adverse effects, including chemotherapy-induced peripheral neuropathy (CIPN) caused by the administration of the chemotherapeutic agent.

### BACKGROUND

It is a priority goal of the National Cancer Institute to find remedies for chemotherapy-induced peripheral neuropathy (CIPN), an often debilitating and frequently treatment-jeopardizing side-effect of many established chemotherapies. Presently, there are no effective countermeasures beyond dose reduction, delay or cessation. The reduced dose exposure that results, risks accelerated tumor growth, chemotherapy resistance, and treatment failure. Moreover, the discomfort of CIPN frequently persists for months to even years beyond the end of treatment, which further degrades quality of life and impedes the capacity for essential follow-on chemotherapy, when needed.

Discovery of a CIPN remedy is particularly crucial for paclitaxel (PTX), one of the most widely-used and efficacious chemotherapy drugs, but also one with a particularly high risk for CIPN, or more specifically, PIPN. The prevalence of PIPN exceeds 60% of patients undergoing PTX infusion. Severe PIPN (grades 3/4), where treatment modifications are indicated, is at least 10% of patients with PIPN.

The symptoms of PIPN include tingling, numbness, burning and pain sensations in the extremities along with loss of effective grip, fine dexterity and balance. The patient experience ranges from annoying to debilitating. The symptoms, their severity, and the amount of peripheral limb axis involved, increase as the size, frequency and number of PTX doses increase. Recovery from PIPN is slow, often interfering with activities of daily living for months to years after chemotherapy completion.

Dose reduction is, currently, the only effective remedy for the painful, debilitating, sensory and motor effects of PIPN. Extended infusion times and treatment intervals yield limited improvements. Potential neuroprotective and symptom-relieving drugs have been evaluated but without significant success.

Both currently approved PTX formulations, Taxol® and Abraxane®, cause PIPN to similar extents and degrees of persistence. The problem is not unique to paclitaxel. PIPN is also a major risk for the other taxanes in clinical use, namely docetaxel (Taxotere®) and cabazitaxel (Jevtana®), and at lower doses.

The success of PTX-based chemotherapy would be greatly enhanced with a formulation technology that sequestered PTX from nerves and targeted PTX to cancerous cells instead. The cancer patient would experience improved treatment outcomes (i.e. better survival) without the discomfort or risk to therapy from PIPN.

### SUMMARY

In a first aspect, the present disclosure provides a method for the treatment of cancer in a subject in need thereof, the method comprising administering a therapeutically effective dose of a composition comprising a chemotherapeutic agent associated with a peptide amphiphile lipid micelle (PALM), wherein the subject experiences reduced chemotherapy-induced peripheral neuropathy when dosed with PALM, than when treated with the chemotherapeutic agent in the absence of treatment with PALM. PALM are formed from a combination of amphiphilic peptide with phospholipids and optionally other hydrophobic molecules, in aqueous suspension.

In a second aspect, the present disclosure provides methods for the treatment of chemotherapy-induced peripheral neuropathy (CIPN) in a subject currently and/or previously treated with a CIPN causing chemotherapeutic agent in need thereof, the method comprising administering a therapeutically effective dose of a composition comprising a chemotherapeutic agent conjugated to a peptide amphiphile lipid micelle (PALM) thus forming PALM nanoparticles, wherein the subject experiences reduced chemotherapy-induced peripheral neuropathy, (e.g. paclitaxel induced peripheral neuropathy) when dosed with PALM nanoparticles, than when treated with the chemotherapeutic agent in the absence of treatment with PALM nanoparticles. In related embodiments, the chemotherapy-induced peripheral neuropathy (CIPN) is caused by and/or associated with taxanes; epothilones (e.g. ixabepilone and sagopilone); vinca alkaloids, e.g. vinblastine, vincristine, vinorelbine, and etoposide (VP-16); thalidomide (Thalomid®), lenalidomide (Revlimid®), and pomalidomide (Pomalyst®); proteasome inhibitors, such as bortezomib (Velcade®), carfilzomib (Kyprolis®), and ixazomib (Ninlaro); topoisomerase inhibitors, such as irinotecan or topotecan; and platinum analogs, including, cisplatin, carboplatin, and oxaliplatin. In related embodiments, the chemotherapy-induced peripheral neuropathy (CIPN) is caused by and/or associated with taxane chemotherapy, for example, treatment of cancer with anyone or more of paclitaxel (Taxol®), Abraxane®; docetaxel (Taxotere®) cabazitaxel (Jevtana®), larotaxel, milataxel, ortataxel, BMS-275183, and tesetaxel.

In a third aspect, the present disclosure provides methods for the treatment of paclitaxel induced peripheral neuropathy (PIPN) in a cancer subject currently and/or previously treated with paclitaxel, the method comprising administering a therapeutically effective dose of a composition comprising PALM nanoparticles, wherein the PALM nanoparticles comprise PALM conjugated to paclitaxel. In some related embodiments, the cancer subject experiences reduced PIPN when dosed with PALM nanoparticles containing paclitaxel, than when treated with paclitaxel alone in the absence of treatment with PALM nanoparticles comprising paclitaxel.

### BRIEF DESCRIPTION OF THE DRAWINGS

FIGS. 1A and 1B are Edmundson Wheel depictions of the peptides of SEQ ID NOs: 3 and 25 respectively showing their amphiphilic conformation. FIGS. 1A and 1B further show the axial positions of the constituent amino acids (identified by standard single letter abbreviations) around the long axis of the alpha-helix. The letter “B” represents 2-amino-isobutyric acid. The dashed lines indicate the approximate boundaries between hydrophilic amino acids (shaded) forming the polar faces of the peptides and the hydrophobic amino acids forming the non-polar faces. FIGS. 1C and 1D are helical net depictions of the peptides of SEQ ID NOs: 3 and 25 respectively.

FIG. 2. The size exclusion chromatogram of PALM containing miriplatin (solid line) compared to human HDL(dashed line). PALM was composed of peptide of SEQ ID NO:25 and POPC, SM and miriplatin at a 2.5:3:7:0.75 mole ratio.

FIG. 3. Shows the size exclusion chromatograph of PALM containing XC and prepared with the peptide of SEQ ID NO:25 at a peptide:phospholipid:XC mole ratio of 1:4:0.4. The elution positions of protein standards of various Stokes diameters are marked.

FIG. 4. Comparison of the size exclusion chromatograms for PALM containing XTT and prepared with peptide of SEQ ID NO:25 (dashed line) or with R4F peptide (solid line). The composition of both was peptide:POPC:SM:XTT at a mole equivalent ratio of 1:2.8:1.2:0.4.

FIG. 5. Depicts the size exclusion chromatogram of PALM prepared with the peptide of SEQ ID NO:25 and containing fenretinide. The PALM composition was peptide:POPC:SM:fenretinide at a mole equivalent ratio of 2.5:3:7:2.

FIG. 6. Inhibition of PC3 prostate cancer cell growth by PALM(MP) compared to inhibition by cisplatin

FIG. 7. Effect of SR-BI antibody on inhibition of PC3 prostate cancer cell growth by PALM(MP). The lines indicate fits of the data to the logistic equation.

FIG. 8. Inhibition of SKOV3 ovarian cancer cell growth by PALM/(XC) (square, dotted line) or PALM(XTT) (diamond, solid line) compared to inhibition by paclitaxel (circle, dashed line) The lines indicate fits of the data to the logistic equation.

FIG. 9. PALM prepared with various peptides, as indicated, and containing DiI, were incubated with BHK(SR-BI) cells that were stably transfected with a mifepristone inducible, human SR-BI gene. The incubations were performed with un-induced (Control) or induced cells. Human HDL, labeled with DiI, was tested for comparison. The amount of DiI taken up by cells over 4 hours of incubation was detected by fluorescence.

FIG. 10. BHK(SR-BI) cells with a mifepristone inducible, human SR-BI gene, which was either induced (SR-BI+) or un-induced (Control), were incubated with the indicated concentrations of PTX or PALM(XTT) for 12 hours Cells were incubated further in the absence of test agents for an additional 36 hours before detection of % growth by MTT assay.

FIG. 11. SR-BI antibody blocks XTT uptake from PALM(XTT) (arrow).

FIG. 12. A bar graph showing the cytokine IL-6 secretion by SKOV-3 cells incubated 24 hours with no addition (control), lipopolysaccharide (10 μg/ml LPS), paclitaxel (PTX), or PALM(XTT).

FIG. 13. A line graph showing human ovarian tumor (SKOV-3) growth in athymic mice injected with Cremophor/ethanol vehicle (A), paclitaxel (10 mg/kg) (B), PALM(XTT) (8 mg/kg paclitaxel equivalents) (c), or PALM(XTT) (24 mg/kg paclitaxel equivalents) (D).

FIG. 14. A line graph showing the mechanical allodynia in rats injected with Cremophor/ethanol vehicle (A), 1 mg/kg paclitaxel (B), saline (PALM vehicle) (C), 1 mg/kg equivalent dose PALM(XTT) (D), 2.7 mg/kg equivalent dose PALM(XTT) (E).

FIG. 15. A line graph showing human ovarian tumor (SKOV-3) growth in athymic mice injected with Cremophor/ethanol vehicle (A), paclitaxel (10 mg/kg) (B), or PALM without XTT (C).

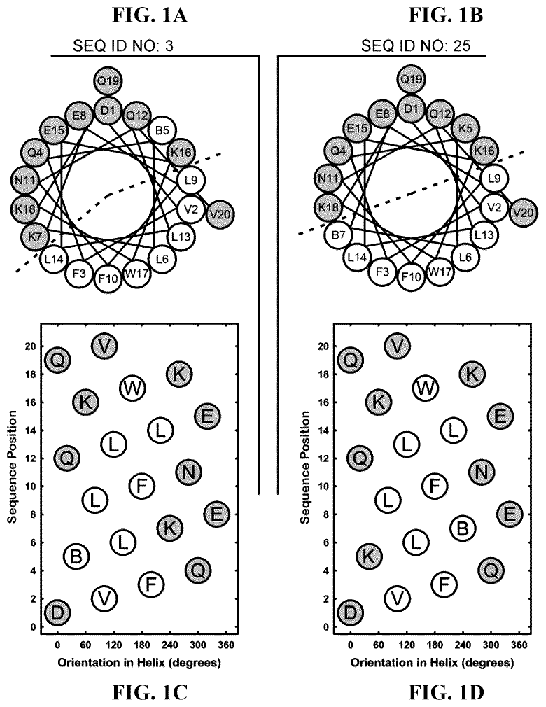

### DETAILED DESCRIPTION

### Definitions

“Nanoparticle” means a particle having no dimension greater than 200 nm.

As used herein, the singular forms “a”, “an” and “the” include plural references unless the context clearly dictates otherwise.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

The antecedent “about” indicates that the values are approximate. For example, the range of “about 1 mg to about 50 mg” indicates that the values are approximate values. The range of “about 1 mg to about 50 mg” includes approximate and specific values, e.g., the range includes about 1 mg. 1 mg, about 50 mg and 50 mg.

When a range is described, the range includes both the endpoints of the range as well as all numbers in between. For example, “between 1 mg and 10 mg” includes 1 mg. 10 mg and all amounts between 1 mg and 10 mg. Likewise, “from 1 mg to 10 mg” includes 1 mg. 10 mg and all amounts between 1 mg and 10 mg.

As used herein, “alkyl” refers to a saturated aliphatic hydrocarbon group containing from 7-21 carbon atoms. As used herein, the terminology (C₁-Cn) alkyl refers to an alkyl group containing 1-n carbon atoms. For example, (C₈-C₁₂) alkyl refers to an alkyl group containing 8, 9, 10, 11, or 12 carbon atoms. An alkyl group can be branched or unbranched.

As used herein, “alkenyl” refers to an aliphatic carbon group that contains from 7-21 carbon atoms and at least one double bond. As used herein, the terminology (C₁-Cn) alkenyl refers to an alkenyl group containing 1-n carbon atoms. An alkenyl group can be branched or unbranched.

“Consisting essentially of” when used to describe the lipid component means that the lipid component includes less than 0.1 mol % of any additional lipid other than those specified.

“XC” is an abbreviation for paclitaxel 2′-cholesteryl carbonate.

“XT3” or “XTT” are abbreviations for paclitaxel 2′-δ-tocotrienyl carbonate.

“MP” is an abbreviation for miriplatin.

“PTX” is an abbreviation for paclitaxel.

“POPC” is an abbreviation for 1-palmitoyl-2-oleoyl phosphatidylcholine.

“SM” is an abbreviation for sphingomyelin.

“TBA” is an abbreviation for tert-butyl alcohol.

“DMSO” is an abbreviation for dimethylsulfoxide

“HDL” is an abbreviation for high density lipoprotein.

“SR-BI” is an abbreviation for scavenger receptor class B, type 1.

“BHK” is an abbreviation for baby hamster kidney.

“DiI” is an abbreviation for 1,1′-dioctadecyl-3,3,3′,3′-tetramethylindocarbocyanine.

“IL-6” is an abbreviation for interleukin-6.

“CIPN” is an abbreviation for chemotherapy-induced peripheral neuropathy.

“PIPN” is an abbreviation for paclitaxel-induced peripheral neuropathy.

“PALM” is an acronym used to identify the peptide-amphiphile lipid micelles formed from a combination of amphiphilic peptide with phospholipids and optionally other hydrophobic molecules, in aqueous suspension.

“Amphiphilic” describes a molecule or polymer (e.g. peptide) with affinity for both lipid and aqueous phases due to a conformation in which hydrophilic (water seeking) substituents and hydrophobic (water avoiding) substituents in the molecule or polymer are structurally segregated from one another.

“Lipophilic” describes a substance that distributes preferentially to lipid domains of lipid-rich particles in aqueous suspension. The lipid-rich particles include lipid micelles, liposomes, lipoproteins, cell membranes and lipid emulsions.

“Peptide” is a polymer produced from alpha-amino acid monomers joined together by amide bonds formed between the carboxylic group of one amino acid and the alpha-amine group of the next amino acid in the polymer. “Peptide” also includes a polymer of amino acid monomers joined together. Both L-optical isomers and the D-optical isomers of amino acids can be used. Amino acids making up the polymer may be either those found in nature (i.e. natural amino acids) or un-natural amino acids. The term “residue” or “amino acid residue” includes reference to an amino acid that is incorporated into a peptide, polypeptide, or protein.

Peptide sequences according to convention, and as used herein, are written N terminus to C-terminus, left to right.

“Micelle” is a multi-molecular structure organized by non-covalent interactions in an aqueous phase. The micelle is composed of amphiphilic and hydrophobic molecules which aggregate in such a manner that the hydrophobic domains of molecules are shielded from the water and the hydrophilic constituents are at the micelle-water interface.

“Cargo molecules” are hydrophobic or amphiphilic chemotherapeutic molecules with anti-cancer therapeutic or diagnostic properties that are stably incorporated into PALM and do not disrupt the stability of PALM.

“siRNA” are small, interfering ribonucleic acids created to control cellular gene expression as part of the RNA-induced gene silencing complex.

“Aib” is the three letter code for the amino acid alpha-amino isobutyric acid.

“Aba” is the three letter code for the amino acid alpha-amino butyric acid.

“Amv” is the three letter code for the unnatural amino acid alpha-methyl valine.

“Orn” is the three letter code for the amino acid ornithine.

“SEC” is size exclusion chromatography.

“DLS” is dynamic light scattering.

The “subject” is defined herein to include animals such as mammals, including, but not limited to, primates (e.g., humans), cows, sheep, goats, horses, dogs, cats, rabbits, rats, mice and the like. In preferred embodiments, the subject is a human. Subject includes cancer patient or cancer patients.

As used herein “chemotherapeutic agent” or “chemotherapy agent” or “antineoplastic agent” refer to an agent that reduces, prevents, and/or delays the growth of metastases or neoplasms, or kills neoplastic cells directly by necrosis or apoptosis in a pharmaceutically-effective amount, to reduce, prevent, and/or delay the growth of metastases or neoplasms in a subject with neoplastic disease.

“Chemotherapy” refers to treatments using chemotherapeutic agents, chemotherapy agents, or antineoplastic agents.

“Effective amount” or a “pharmaceutically-effective amount” in reference to the composition containing PALM conjugated to a chemotherapeutic agent refers to the amount of said composition sufficient to induce a desired biological, pharmacological, or therapeutic outcome in a subject.

“Chemotherapy-induced peripheral neuropathy” is a toxic neuropathy that results from the direct injury of the peripheral nervous system by a chemotherapeutic agent(s). CIPN can be acute or chronic. CIPN can be sensory, motor, autonomic, or a mixture of any of the three classes.

“Neurotoxic effects” and “neurotoxicity” refers to toxic substances altering the normal activity of the nervous system.

“Neuropathic pain” is the intractable pain caused by dysfunction in the peripheral or central nervous system.

Without wishing to be bound by any theory, it is believed that paclitaxel (PTX) interferes with nerve function by several mechanisms. The most prominent is a perineuronal inflammation resulting from the capacity of PTX to activate the toll-like receptor 4 (TLR4) in resident macrophages (microglia) adjacent to nerves. TLR4 binding by PTX prompts the production and release of inflammatory cytokines by the microglia. The cytokines then go on to activate pain channels in adjacent nerves. PIPN also stems from PTX that enters nerves and, because of its tubulin targeting capacity, interferes with tubulin-dependent neurotransmitter transport in axons. Further, there is evidence that PTX causes nerve atrophy and loss.

Paclitaxel is also at the root of cognitive impairment, another neurological disorder plaguing chemotherapy patients. Paclitaxel-induced cognitive impairment results from the ability of paclitaxel to infiltrate the hippocampus and cause inflammation and interfere with neuron function there. These are the same processes responsible for PIPN.

The multiple mechanisms by which PTX impacts nerves, suggests a single symptom-relieving medicament to counteract PIPN is unlikely to succeed. A better approach would be development of a formulation technology that sequesters PTX from TLR4 and nerves altogether while maintaining exposure of tumors.

There are added benefits for a technology that restrains PTX from TLR4 interaction. PTX interaction with TLR4 is linked to gastrointestinal inflammation and chemo-resistance. Activation is also linked to induction of cancer cell metastasis and growth. Furthermore, the inflammation cascade triggered by TLR4 activation leads to immune-suppression within the tumor. It has been shown in mouse models that inflammation induced by PTX leads to decreased immunosuppression of tumor growth. These examples of the influence of TLR4 on tumor progression are supported by the observation that tumors with low TLR4 are associated with much greater patient survival.

The present disclosure addresses this need by providing novel PALM nanoparticle formulations of lipid and peptide and methods to form them that allow incorporation of chemotherapeutic molecules, e.g., drugs, and wherein the nanoparticles are stable in infusion or injection solutions. The formulations of the invention provide one or more improvements, including but not limited to, improved pharmacokinetic parameters, increased half-life, targeted delivery, diminished toxicity or an improved therapeutic index for parenterally-administered anti-cancer drugs, in particular for chemotherapeutic agents that cause or are associated with CIPN and in particular PIPN.

The present disclosure provides amphiphilic, alpha-helical peptides that comprise an amino acid sequence of SEQ ID NO: 1, SEQ ID NO:24, SEQ ID NO:37 or SEQ ID NO:59.

Further, the present disclosure provides peptide amphiphile lipid micelles (PALM) which comprise a peptide comprising an amino acid sequence of the disclosure, sphingomyelin and one or more additional phospholipids. The PALM of the present disclosure optionally comprise one or more cargo molecules, such as imaging agents and drugs.

The present disclosure also provides for processes for preparing PALM and PALM composition formulated with cargo molecules.

Additionally, the present disclosure provides for compound conjugates and methods of preparing compound conjugates suitable for use with PALM.

Further the present disclosure provides for methods of treating or preventing CIPN (e.g. PIPN) adverse events by administering PALM-chemotherapeutic agent conjugates.

The present invention provides a method of treating CIPN in a subject, comprising administering to the subject a therapeutically effective amount of a composition containing PALM nanoparticles containing a CIPN causing chemotherapeutic agent as exemplified herein.

In another aspect, the present invention provides a method for prophylactic treatment of CIPN in a subject, comprising administering to the subject an effective amount of a composition containing PALM nanoparticles containing a CIPN causing chemotherapeutic agent as exemplified herein

In another aspect, the present invention provides a method for mitigating neurotoxic effects of a chemotherapeutic agent which causes and/or is associated with CIPN, comprising administering to a subject an effective amount of a composition containing a PALM nanoparticles containing a CIPN causing chemotherapeutic agent as exemplified herein.

In yet another aspect, the present invention provides a method for treating chemotherapy-induced neuropathic pain in a subject, comprising administering to the subject an effective amount of a composition containing a PALM moiety conjugated to a CIPN causing chemotherapeutic agent as exemplified herein.

In some embodiments of the present disclosure, the PALM moiety contains or comprises one or more “amphiphilic peptides”. Amphiphilic peptides are able to adopt an alpha helical conformation in which the helix has opposing polar and non-polar faces oriented along the long axis of the helix. Techniques of synthesizing peptides are well known in the art. The peptides of the present disclosure can be synthesized by any technique known in the art.

Table 1 shows the charge distribution of specific amphiphilic peptides of the present disclosure compared with several prior art sequences. The charge distribution of the peptides of the present invention are novel in view of the prior art shown below.

TABLE 1
Charges of Residues in Apolipoprotein A-I Mimetic Peptides at Neutral pH

| | N-Term | Amino Acid Position | | | | | | | | | | | | | | | | | | | | | | | | C-Term |
|---|---|---|---|---|---|---|---|---|---|---|---|---|---|---|---|---|---|---|---|---|---|---|---|---|---|---|
| Peptide | Charge | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | Charge |
| SEQ ID^{a} | + | − | ∘ | ∘ | ∘ | ∘ | ∘ | + | − | ∘ | ∘ | ∘ | ∘ | ∘ | ∘ | − | + | ∘ | + | ∘ | ∘ | | | | | − |
| SEQ ID^{b} | + | − | ∘ | ∘ | ∘ | + | ∘ | ∘ | − | ∘ | ∘ | ∘ | ∘ | ∘ | ∘ | − | + | ∘ | + | ∘ | ∘ | | | | | − |
| A-Icon^{c} | + | ∘ | ∘ | ∘ | − | − | ∘ | + | − | + | ∘ | ∘ | − | ∘ | ∘ | − | ∘ | ∘ | + | ∘ | + | ∘ | + | | | − |
| LAP642^{d} | + | ∘ | ∘ | ∘ | − | ∘ | ∘ | + | − | ∘ | ∘ | ∘ | − | ∘ | ∘ | − | ∘ | ∘ | + | ∘ | + | ∘ | + | | | − |
| 18A ^{e} | + | − | ∘ | ∘ | + | ∘ | ∘ | ∘ | − | + | ∘ | ∘ | − | + | ∘ | + | − | ∘ | ∘ | | | | | | | − |
| 2F ^{f} | ∘ | − | ∘ | ∘ | + | ∘ | ∘ | ∘ | − | + | ∘ | ∘ | − | + | ∘ | + | − | ∘ | ∘ | | | | | | | ∘ |
| R4F ^{g} | ∘ | ∘ | ∘ | − | + | ∘ | + | − | ∘ | ∘ | + | − | ∘ | ∘ | ∘ | + | ∘ | ∘ | − | | | | | | | ∘ |
| FAMP ^{h} | + | ∘ | ∘ | − | ∘ | ∘ | ∘ | ∘ | ∘ | ∘ | − | + | ∘ | ∘ | + | ∘ | ∘ | − | − | ∘ | ∘ | + | + | ∘ | ∘ | − |

^{a}SEQ ID NOs: 1-23
^{b}SEQ ID NOs: 24-35
^{c}Anantharamaiah et al. (1990) Arteriosclerosis 10: 95-105
^{d}Homan et al. (2013) Anal. Biochem. 441: 80-86
^{e} Anantharamaiah et al. (1985) J. Biol. Chem. 260: 10248-10255
^{f} Datta et al. (2001) J. Lipid Res. 42: 1096-1104
^{g} Zhang et al. (2009) Angew. Chem. Int. Ed. 48: 9171-9175
^{h} Uehara et al. (2013) J Am Heart Assoc. 2(3): e000048. doi: 10.1161/JAHA.113.000048
“∘” indicates zero charge at the indicated position.
“+” indicates a positive charge at the indicated position.
“−” indicates a negative charge at the indicated position.

One embodiment of the first aspect of the disclosure provides a peptide that comprises the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈- X₁₉-X₂₀ wherein: X₁ is the amino acid D; X₂ and X₂₀ are each the amino acid V or Aib; X₃, X₆, X₁₀ and X₁₃ are each an amino acid independently selected from the group consisting of L and F; X₄, X₁₂ and X₁₉ are each the amino acid Q; X₅ is an amino A or Aib; X₇, X₁₆ and X₁₈ are each the amino acid K; X₈ and X₁₅ are each the amino acid E; X₉ and X₁₄ are each an amino acid independently selected from the group consisting of A, L, F and Aib; X₁₁ is an amino acid selected from the group consisting of A, Aib and N; and X₁₇ is an amino acid selected from the group consisting of W, F and L, (SEQ ID NO:1) wherein the peptide is from 20 to 24 amino acid in length.

Another embodiment of the first aspect provides a peptide that consists essentially of the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈- X₁₉-X₂₀ wherein: X₁ is the amino acid D; X₂ and X₂₀ are each the amino acid V or Aib; X₃, X₆, X₁₀ and X₁₃ are each an amino acid independently selected from the group consisting of L and F; X₄, X₁₂ and X₁₉ are each the amino acid Q; X₅ is an amino A or Aib; X₇, X₁₆ and X₁₈ are each the amino acid K; X₈ and X₁₅ are each the amino acid E; X₉ and X₁₄ are each an amino acid independently selected from the group consisting of A, L, F and Aib; X₁₁ is an amino acid selected from the group consisting of A, Aib and N; and X₁₇ is an amino acid selected from the group consisting of W, F and L, (SEQ ID NO:1) wherein the peptide is from 20 to 24 amino acid in length.

Still another embodiment of the first aspect provides a peptide that consists of the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉- X₂₀ wherein: X₁ is the amino acid D; X₂ and X₂₀ are each the amino acid V or Aib; X₃, X₆, X₁₀ and X₁₃ are each an amino acid independently selected from the group consisting of L and F; X₄, X₁₂ and X₁₉ are each the amino acid Q; X₅ is an amino A or Aib; X₇, X₁₆ and X₁₈ are each the amino acid K; X₈ and X₁₅ are each the amino acid E; X₉ and X₁₄ are each an amino acid independently selected from the group consisting of A, L, F and Aib; X₁₁ is an amino acid selected from the group consisting of A, Aib and N; and X₁₇ is an amino acid selected from the group consisting of W, F and L. (SEQ ID NO:1)

Yet another embodiment of the first aspect provides a peptide that comprises the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉- X₂₀ wherein: X₁, X₈ and X₁₅ are independently selected from the group consisting of the amino acids D and E; X₂ and X₂₀ are each an amino acid independently selected from the group consisting of V, I, Aib, and L; X₃, and X₁₃ are each an amino acid independently selected from the group consisting of L, I, V, W, Y, Amv, Aib, and F; X₆ and X₁₀ are each an amino acid independently selected from the group consisting of L, I, V, W, Y, Aib, Amv, and F; X₄, X₁₂ and X₁₉ are each an amino acid independently selected from the group consisting of Q and N; X₅, X₁₆ and X₁₈ are each an amino acid independently selected from the group consisting of K, R, H and Orn; X₇ is an amino acid selected from A, G, S, Aib and Aib; X₉, and X₁₄ are each an amino acid independently selected from the group consisting of A,G, S, V, Amv, and Aib; X₁₁ is an amino acid selected from the group consisting of A, G, S, Aib, Amv, V and N; X₁₇ is an amino acid selected from the group consisting of W, F, Y, I, V, and L, (SEQ ID NO:24) wherein the peptide is from 20 to 24 amino acid in length.

Another embodiment of the first aspect provides a peptide that consists essentially of the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈- X₁₉-X₂₀ wherein: X₁ is an amino acid selected from the group consisting of D and E; X₂ and X₂₀ are each an amino acid independently selected from the group consisting of V, I, Aib, and L; X₃, X₆, X₁₀ and X₁₃ are each an amino acid independently selected from the group consisting of L, I, V, W, Y, Aib, Amv and F; X₄, X₁₂ and X₁₉ are each an amino acid independently selected from the group consisting of Q and N; X₅, X₁₆ and X₁₈ are each an amino acid independently selected from the group consisting of K, R, H and Orn; X₇ is selected from the group consisting of A, G, S, V, Aib and Amv; X₈ and X₁₅ are independently selected from the group consisting of the amino acid E and D; X₉ and X₁₄ are an amino acid independently selected from the group consisting of A, G, S L, F, V, Amv, and Aib; X₁₁ is an amino acid selected from the group consisting of A, G, S, Aib, Amv, V and N; and X₁₇ is an amino acid selected from the group consisting of W, F, Y, I, V, and L, (SEQ ID NO:24) and the peptide is from 20 to 24 amino acid in length.

Still another embodiment of the first aspect provides a peptide that consists of the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉- X₂₀ wherein: X₁ is an amino acid selected from the group consisting of D and E; X₂ and X₂₀ are each an amino acid independently selected from the group consisting of V, I, Aib and L; X₃, X₆, X₁₀ and X₁₃ are each an amino acid independently selected from the group consisting of L, I, V, W, Y, Aib, Amv and F; X₄, X₁₂ and X₁₉ are each an amino acid independently selected from the group consisting of Q and N; X₅, X₁₆ and X₁₈ are each an amino acid independently selected from the group consisting of K, R, H and Orn; X₇ is selected from the group consisting of A, G, S, V, Aib and Amv; X₈ and X₁₅ are independently selected from the group consisting of the amino acid E and D; X₉ and X₁₄ are an amino acid independently selected from the group consisting of A, G, S L, F, V, Amv, and Aib; X₁₁ is an amino acid selected from the group consisting of A, G, S, Aib, Amv, V and N; and X₁₇ is an amino acid selected from the group consisting of W, F, Y, I, V, and L. (SEQ ID NO:24).

It is contemplated that any of the disclosed embodiments of the peptides according to the first aspect are optionally acylated at the alpha-amine of the N-terminal amino acid of the peptide, optionally amidated at the terminal carboxyl group of the peptide, or optionally acylated at the alpha-amine of the N-terminal amino acid and amidated at the terminal carboxyl group of the peptide. Peptides can be acylated or amidated by methods known in the art.

Particular peptides of the present invention are provided in Table 2 below.

TABLE 2

| SEQ | | Mean | Mean |
|---|---|---|---|
| ID | | Hydrophobic | Hydro- |
| NO: | Peptide Sequence | Moment^{a} | phobicity^{b} |
| 2 | DVFQALKELFAQLLEKWKQV | 0.846 | −1.043 |
| 3 | DVFQ{AIB}LKELFNQLLEKWKQV | 0.908 | −1.135 |
| 4 | DVFQ{AIB}LKELLAQLLEKFKQV | 0.885 | −0.995 |
| 5 | DVFQ{AIB}LKELLNQLLEKFKQV | 0.948 | −1.092 |
| 6 | DVFQ{AIB}LKELLNQL{AIB}EKFKQV | 0.940 | −1.120 |
| 7 | DVFQ{AIB}LKELLNQL{AIB}EKWKQV | 0.910 | −1.151 |
| 8 | DVFQALKELLAQLLEKFKQV | 0.887 | −1.000 |
| 9 | DVFQALKELLNQLLEKFKQV | 0.950 | −1.097 |
| 10 | DVFQ{AIB}LKELFAQLLEKWKQV | 0.845 | −1.038 |
| 11 | DVFQ{AIB}LKELFNQLLEKWKQV | 0.908 | −1.135 |
| 12 | DVFQ{AIB}LKELFNQLLEKFKQV | 0.938 | −1.104 |
| 13 | DVFQALKELFAQL{AIB}EKWKQV | 0.836 | −1.071 |
| 14 | DVFQALKELFNQL{AIB}EKWKQV | 0.902 | −1.168 |
| 15 | DVFQALKELFNQL{AIB}EKFKQV | 0.932 | −1.137 |
| 16 | DVFQAFKEAFAQLFEKWKQV | 0.821 | −1.099 |
| 17 | DVFQAFKE{AIB}FAQLFEKWKQV | 0.822 | −1.094 |
| 18 | DVFQ{AIB}FKE{AIB}FAQLFEKWKQV | 0.820 | −1.089 |
| 19 | DVFQAFKEAF{AIB}QLFEKWKQV | 0.818 | −1.094 |
| 20 | DVFQAFKE{AIB}F{AIB}QLFEKWKQV | 0.819 | −1.089 |
| 21 | DVFQ{AIB}FKE{AIB}F{AIB}QLFEKWKQV | 0.817 | −1.084 |
| 22 | DVFQALKELFNQLLEKWKQV | 0.910 | −1.140 |
| 23 | DVFQ{AIB}LKELLNQLLEKLKQV | 0.959 | −1.081 |
| 25 | DVFQKL{AIB}ELFNQLLEKWKQV | 0.976 | −1.135 |
| 26 | DVFQKLVELFNQLLEKWKQV | 0.979 | −1.119 |
| 27 | DV{AIB}QKLFELFNQLLEKWKQV | 0.966 | −1.135 |
| 28 | DVFQKL{AIB}ELFNQLLEKFKQV | 1.007 | −1.104 |
| 29 | DVFQKLVELFNQLLEKFKQV | 1.010 | −1.088 |
| 30 | DV{AIB}QKLFELFNQLLEKFKQV | 0.997 | −1.104 |
| 31 | DVLQKF{AIB}ELFNQLLEKWKQV | 0.974 | −1.135 |
| 32 | DV{AIB}QKFLELFNQLLEKWKQV | 0.958 | −1.135 |
| 33 | DVFQKLLE{AIB}FNQLLEKWKQV | 0.979 | −1.135 |
| 34 | DVFQKL{AIB}ELFNQ{AIB}LEKWKQV | 0.955 | −1.163 |
| 35 | DVFQKL{AIB}ELFNQL{AIB}EKWKQV | 0.961 | −1.163 |
| 36 | D{AIB}FQKL{AIB}ELFNQL{AIB}EKWKQV | 0.953 | −1.179 |

^{a}Calculated from amino acid hydrophobicity (Hessa et al. Nature 433:377-381 (2005))according to Pownall et al. (FEBS Letters 159:17-23 (1983)).
^{b}Calculated as the sum of amino acid hydrophobicities divided by the number of residues (kcal/mol/residue).

One embodiment of the first aspect of the disclosure is a peptide comprising any one of the amino acid sequences of SEQ ID NOs: 1-23 where the peptide is from 20 to 24 amino acid in length. Yet another embodiment is a peptide consisting essentially of any one of the amino acid sequences of SEQ ID NOs: 1-23 where the peptide is from 20 to 24 amino acid in length. Yet another embodiment is a peptide consisting of any one of the amino acid sequences of SEQ ID NOs: 1-23. In any of the above embodiments of the disclosed peptides, optionally the alpha-amine of the N-terminal amino acid of the peptide is acylated; the terminal carboxyl group is amidated; or the alpha-amine of the N-terminal amino acid is acylated and the terminal carboxyl group of the peptide is amidated.

One embodiment of the first aspect of the disclosure is a peptide comprising any one of the amino acid sequences of SEQ ID NOs: 25-36 where the peptide is from 20 to 24 amino acid in length. Still another embodiment is a peptide consisting essentially of any one of the amino acid sequences of SEQ ID NOs: 25-36 where the peptide is from 20 to 24 amino acid in length. Another embodiment is a peptide consisting of any one of the amino acid sequences of SEQ ID NOs: 25-36. In any of the above embodiments of the peptides, optionally the alpha-amine of the N-terminal amino acid of the peptide is acylated; the terminal carboxyl group is amidated; or the alpha-amine of the N-terminal amino acid is acylated and the terminal carboxyl group of the peptide is amidated.

Embodiments of the present disclosure further include peptides that have the reverse sequence of the peptides generically defined by SEQ ID NOs: 1 and 24.

One embodiment of the first aspect of the disclosure provides peptides that are the reverse of SEQ ID NO: 1 and the peptides comprise the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀, wherein X₁ and X₁₉ are each the amino acid V or Aib; X₂, X₉ and X₁₇ are each the amino acid Q; X₃, X₅, and X₁₄ are each the amino acid K; X₄ is an amino acid selected from the group consisting of W, F, and L; X₆ and X₁₃ are each the amino acid E; X₇ and X₁₂ are each an amino acid independently selected from the group consisting of A, L, F, and Aib; X₈, X₁₁, X₁₅ and X₁₈ are each an amino acid independently selected from the group consisting of L and F; X₁₀ is an amino acid selected from the group consisting of A, Aib and N; X₁₆ is an amino acid selected from the group consisting of A and Aib; and X₂₀ is the amino acid D. (SEQ ID NO:37), wherein the peptide is from 20 to 24 amino acid in length.

Another embodiment of the first aspect of the disclosure provides peptides that are the reverse of SEQ ID NO:24 and the reverse peptides comprise the amino acid sequence: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀ wherein X₁ and X₁₉ are each an amino acid independently selected from the group consisting of V, Aib, I and L; X₂, X₉ and X₁₇ are each an amino acid independently selected from the group consisting of Q and N; X₃, X₅, and X₁₆ are each an amino acid independently selected from the group consisting of K, R, H, and Orn; X₄ is an amino acid selected from the group consisting of W, F, Y, I, V, and L; X₆, X₁₃ and X₂₀ are each an amino acid independently selected from the group consisting of E and D; X₇ and X₁₂ are each an amino acid independently selected from the group consisting of A, G, S, L, F, V, Amv and Aib; X₈, X₁₁, X₁₅, and X₁₈ are independently selected from the group consisting of the amino acid L, I, V, W, and F; X₁₀ is an amino acid selected from the group consisting of A, G, S, Aib, Amv, V and N; and X₁₄ is an amino acid selected from the group consisting of A, G, S, V, Aib and Amv. (SEQ ID NO:59), wherein the peptide is from 20 to 24 amino acid in length.

Another embodiment of the first aspect of the disclosure provides peptides that are the reverse of SEQ ID NO:24 and the reverse peptides consist of the amino acid sequence: X₁-X₂-X₃-X₄-X₅-X₆-X₇-X₈-X₉-X₁₀-X₁₁-X₁₂-X₁₃-X₁₄-XIS-X₁₆-X₁₇-X₁₈-X₁₉-X₂₀, wherein X₁ and X₁₉ are each the amino acid V or Aib; X₂, X₉ and X₁₇ are each the amino acid Q; X₃, X₅, and X₁₄ are each the amino acid K; X₄ is an amino acid selected from the group consisting of W, F, and L; X₆ and X₁₃ are each the amino acid E; X₇ and X₁₂ are each an amino acid independently selected from the group consisting of A, L, F, and Aib; X₈, X₁₁, X₁₅ and X₁₈ are each an amino acid independently selected from the group consisting of L and F; X₁₀ is an amino acid selected from the group consisting of A, Aib and N; X₁₆ is an amino acid selected from the group consisting of A and Aib; and X₂₀ is the amino acid D. (SEQ ID NO:37).

Provided in Table 3 are additional peptides of the present invention. The sequences of the amino acids in these peptides are the reverse of the amino acid sequences of SEQ ID NOs:2-23 and 25-36.

TABLE 3

| SEQ | |
|---|---|
| ID NO: | Peptide Sequence |
| 38 | VQKWKELLQAFLEKLAQFVD |
| 39 | VQKWKELLQNFLEKL{AIB}QFVD |
| 40 | VQKFKELLQALLEKL{AIB}QFVD |
| 41 | VQKFKELLQNLLEKL{AIB}QFVD |
| 42 | VQKFKE{AIB}LQNLLEKL{AIB}QFVD |
| 43 | VQKWKE{AIB}LQNLLEKL{AIB}QFVD |
| 44 | VQKFKELLQALLEKLAQFVD |
| 45 | VQKFKELLQNLLEKLAQFVD |
| 46 | VQKWKELLQAFLEKL{AIB}QFVD |
| 47 | VQKWKELLQNFLEKL{AIB}QFVD |
| 48 | VQKFKELLQNFLEKL{AIB}QFVD |
| 49 | VQKWKE{AIB}LQAFLEKLAQFVD |
| 50 | VQKWKE{AIB}LQNFLEKLAQFVD |
| 51 | VQKFKE{AIB}LQNFLEKLAQFVD |
| 52 | VQKWKEFLQAFAEKFAQFVD |
| 53 | VQKWKEFLQAF{AIB}EKFAQFVD |
| 54 | VQKWKEFLQAF{AIB}EKF{AIB}QFVD |
| 55 | VQKWKEFLQ{AIB}FAEKFAQFVD |
| 56 | VQKWKEFLQ{AIB}F{AIB}EKFAQFVD |
| 57 | VQKWKEFLQ{AIB}F{AIB}EKF{AIB}QFVD |
| 58 | VQKWKELLQNFLEKLAQFVD |
| 60 | VQKLKELLQNLLEKL{AIB}QFVD |
| 61 | VQKWKELLQNFLE{AIB}LKQFVD |
| 62 | VQKWKELLQNFLEVLKQFVD |
| 63 | VQKWKELLQNFLEFLKQ{AIB}VD |
| 64 | VQKFKELLQNFLE{AIB}LKQFVD |
| 65 | VQKFKELLQNFLEVLKQFVD |
| 66 | VQKFKELLQNFLEFLKQ{AIB}VD |
| 67 | VQKWKELLQNFLE{AIB}FKQLVD |
| 68 | VQKWKELLQNFLELFKQ{AIB}VD |
| 69 | VQKWKELLQNF{AIB}ELLKQFVD |
| 70 | VQKWKEL{AIB}QNFLE{AIB}LKQFVD |
| 71 | VQKWKE{AIB}LQNFLE{AIB}LKQFVD |
| 72 | VQKWKE{AIB}LQNFLE{AIB}LKQF{AIB}D |

One embodiment of the first aspect of the disclosure is a peptide comprising any one of the amino acid sequences of SEQ ID NOs: 38-58 where the peptide is from 20 to 24 amino acid in length. Yet another embodiment is a peptide consisting essentially of any one of the amino acid sequences of SEQ ID NOs: 38-58. Yet another embodiment is a peptide consisting of any one of the amino acid sequences of SEQ ID NOs: 38-58. In any of the above embodiments of the peptides, optionally the alpha-amine of the N-terminal amino acid of the peptide is acylated; the terminal carboxyl group is amidated; or the alpha-amine of the N-terminal amino acid is acylated and the terminal carboxyl group of the peptide is amidated.

One embodiment of the first aspect of the disclosure is a peptide comprising any one of the amino acid sequences of SEQ ID NOs: 60-72 where the peptide is from 20 to 24 amino acids in length. Still another embodiment is a peptide consisting essentially of any one of the amino acid sequences of SEQ ID NOs: 60-72. Yet another embodiment is a peptide consisting of any one of the amino acid sequences of SEQ ID NOs: 60-72. In any of the above embodiments of the peptides. optionally the alpha-amine of the N-terminal amino acid of the peptide is acylated; the terminal carboxyl group is amidated; or the alpha-amine of the N-terminal amino acid is acylated and the terminal carboxyl group of the peptide is amidated.

Where a peptide of the disclosure comprises an amino acid sequence of any one of SEQ ID NOs: 1-72 and from 1-4 additional amino independently added to either the N-terminus or C-terminus of the amino acid sequence, the additional amino acids are selected such that the addition of the amino acids does not negatively affect the amphilicity of the peptide.

A second aspect of the disclosure provides a peptide-amphiphile lipid micelle (PALM) moiety (also referred to herein as “PALM”) formed from a combination of amphiphilic peptide with phospholipids. PALM of the second aspect of the disclosure comprise one or more peptides of the first aspect of the disclosure complexed with a lipid component where the lipid component comprises sphingomyelin and one or more additional phospholipids. PALM according to the present disclosure may be passively or actively delivered to a target cell population. In one embodiment of the second aspect of the disclosure. PALM comprises one or more peptides of the present disclosure where the lipid component consists essentially of sphingomyelin and one or more additional phospholipids. In one embodiment PALM comprises a peptide of the present disclosure and a lipid component wherein the lipid component comprises sphingomyelin and one or more additional phospholipids where the additional phospholipid is selected from the group consisting of phosphatidylcholine, polyethylene glycol-phosphatidylethanolamine (PEG-PE), phosphatidylethanolamine, phosphatidylglycerol, phosphatidylserine, phosphatidylinositol, cardiolipin, and any combination thereof. In another embodiment the PALM comprises a peptide of the disclosure and the lipid component comprises sphingomyelin, and phosphatidylcholine. In another embodiment the PALM comprises a peptide of the disclosure, sphingomyelin, and 1-palmitoyl-2-oleoyl phosphatidylcholine (POPC). In yet another embodiment the PALM comprises a peptide of the disclosure and the lipid component comprises sphingomyelin, and phosphatidylethanolamine. In yet another embodiment the PALM comprises a peptide of the disclosure, and the lipid component comprises sphingomyelin, and poly(ethylene glycol)phosphatidyl-ethanolamine. In still another embodiment the PALM comprises a peptide of the disclosure and the lipid component comprises sphingomyelin, and phosphatidylserine. In another embodiment the PALM comprises a peptide of the disclosure and the lipid component comprises sphingomyelin and cardiolipin.

In still another embodiment of the second aspect of the disclosure, PALM comprises a peptide of the disclosure and the lipid component consists essentially of sphingomyelin and one or more additional phospholipid where the one or more additional phospholipid is selected from the group consisting of phosphatidylcholine, polyethylene glycol-phosphatidyl-ethanolamine (PEG-PE), phosphatidylethanolamine, phosphatidylglycerol, phosphatidylserine, phosphatidylinositol, cardiolipin, and any combination thereof. In still another embodiment the PALM comprises a peptide of the disclosure and the lipid component consists essentially of sphingomyelin and phosphatidylcholine. In another embodiment the PALM comprises a peptide of the disclosure and the lipid component consists essentially of sphingomyelin and 1-palmitoyl-2-oleoyl-phosphatidylcholine (POPC).

In some embodiments of the second aspect of the disclosure, PALM comprises a peptide of the disclosure and the lipid component consists essentially of sphingomyelin and one or more additional phospholipid where the one or more additional phospholipid is selected from the group consisting of phosphatidylcholine, polyethylene glycol-phosphatidylethanolamine (PEG-PE), phosphatidylethanolamine, phosphatidylglycerol, phosphatidylserine, phosphatidylinositol, cardiolipin, and any combination thereof where the molar ratio of phospholipid to sphingomyelin is from about 95:5 to about 10:90. In another embodiment the molar ratio of phospholipid to sphingomyelin is from about 90:10 to about 20:80. In still another embodiment the molar ratio of phospholipid to sphingomyelin is from about 25:75 to about 35:65. In another embodiment the molar ratio of phospholipid to sphingomyelin is about 30:70. In another embodiment the molar ratio of phospholipid to sphingomyelin is from about 80:20 to about 60:40. In yet another embodiment the molar ratio of phospholipid to sphingomyelin is from about 75:25 to about 65:35. In still another embodiment the molar ratio of phospholipid to sphingomyelin is about 70:30.

The fatty acid constituents of the phospholipids include fatty acids according to the formula: R—COOH, wherein R is a (C7-C21) alkyl group or a (C7-C21) alkenyl group wherein the alkenyl group can have from one to six double bonds. Examples of suitable fatty acids include, but are not limited to, phytanic acid, linolenic acid, linoleic acid, docosatetraenoic acid, oleic acid, caprylic acid, lauric acid, arachidic acid, myristic acid and palmitic acid. The pair of fatty acids esterified to the glycerol backbone of a particular phospholipid may be identical or each may be a different type of fatty acid.

The molar ratio of the lipid component to peptide is from about 10:1 to about 2:1. In one embodiment the ration is from about 9:1 to about 2:1. In one embodiment the molar ratio of the lipid component to peptide is from about 8:1 to about 2:1. In still another embodiment the molar ratio of the lipid component to peptide is from about 7:1 to about 3:1. In another embodiment the molar ratio of the lipid component to peptide is from about 6:1 to about 4:1.

Complexes of phosphatidylcholine with amphiphilic peptides are known. One method to produce these complexes is by initial co-lyophilization from a common solvent phase followed by rehydration of the dry lyophilizate to form complexes in aqueous suspension.

Particle size is measured by DLS and is expressed as the hydrodynamic mean diameter (“mean diameter”). PALM according to the second aspect of the disclosure are nanometer-sized particles having a mean diameter of 200 nm or less, 50 nm or less, 40 nm or less, or 30 nm or less. In one embodiment the mean particle diameter is from about 5 nm to about 200 nm. In another embodiment the mean particle diameter is from about 5 nm to about 50 nm. In one embodiment the mean particle diameter is from about 5 nm to about 30 nm. In yet another embodiment the mean particle diameter is from about 7.5 nm to about 30 nm. In still another embodiment the mean particle diameter is from about 10 nm to about 30 nm. In another embodiment the mean particle diameter is from about 5 nm to about 25 nm. In another embodiment the mean particle diameter is from about 7.5 nm to about 25 nm. In yet another embodiment the mean particle diameter is from about 10 nm to about 25 nm. In another embodiment the mean particle diameter is from about 5 nm to about 20 nm. In another embodiment the mean particle diameter is from about 7.5 nm to about 20 nm. In yet another embodiment the mean particle diameter is from about 10 nm to about 20 nm. In still another embodiment the mean particle diameter is from about 5 nm to about 15 nm. In another embodiment the mean particle diameter is from about 7.5 nm to about 15 nm. In yet another embodiment the mean particle diameter is from about 10 nm to about 15 nm. In still another embodiment the mean particle diameter is from about 7.5 nm to about 10 nm.

A third aspect of the disclosure provides for PALM-cargo molecule compositions which comprise any one of the PALM embodiments of the second aspect of the disclosure and a cargo molecule. Cargo molecules include, but are not limited to, molecules having pharmaceutical or therapeutic properties. Non-limiting examples of cargo molecules include anti-cancer compounds such as all-trans retinoic acid, alcohol esters of all-trans retinoic acid including methyl, ethyl, and longer chain fatty alkyl chain alcohol esters of retinoic acid and cholesteryl esters of retinoic acid; retinoic acid amides such as fenretinide; retinol and carboxylic acid esters of retinol including methyl, ethyl, and longer chain fatty alkyl chain alcohol esters of retinoic acid; lipophilic anti-fungal agents such as amphotericin B or nystatin; steroids such as progesterone, testosterone, prednisolone, hydrocortisone, dexamethasone and estradiols; analgesics such as propofol and haloperidol; antipsychotics such as fluphenazine decanoate and aripiprazole; the vitamin D analogs cholecalciferol and ergocalciferol; and the isomers of vitamin E, either collectively or individually.

Cargo molecules also include molecules enabling diagnostic or imaging procedures such as fluorescent imaging agents, radiolabeled imaging agents, and agents used for MRI, PET, CT, SPECT/CT and x-ray studies. MRI imaging agents include, but are not limited to, contrast agents such as a phosphatidylethanolamine with a diethylenetriamine pentaacetic acid moiety that is chelated with a gadolinium ion or similar lanthanide ion or indium-111 or gallium-67 or lutetium-177 or samarium-153.

Cargo molecules may also be various types and lengths of RNA or DNA that have been linked to cholesterol or other polycyclic fatty alcohols by known methods.

In one embodiment of the third aspect, the cargo molecule is miriplatin which has the chemical name: cis-[((1R, 2R)-1,2-cyclohexanediamine-N,N′)bis(myristato)] platinum(II).

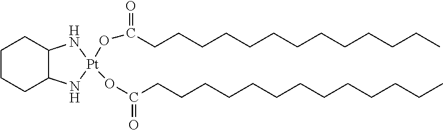

Yet another embodiments of the third aspect of the disclosure, is a PALM-cargo molecule complex wherein the cargo molecule is a compound conjugate of formula I

A—R—L—X (formula I)

wherein A is an agent having an hydroxy or amine group; R is a hydroxyl or an amine group of the agent; L is a linker, and X is an anchor moiety.

Another embodiment of the third aspect of the disclosure is a PALM-cargo molecule complex wherein the cargo molecule is a compound conjugate of formula I:

A—R—L—X (formula I)

wherein A is an agent having a hydroxy or amine group; R is the hydroxyl or the amine group of the agent; L is carbonic acid, succinic acid or diglycolic acid; and X is cholesterol, α-tocotrienol, β-tocotrienol, γ-tocotrienol, δ-tocotrienol, coprostanol, plant sterols, (ß-sitosterol, sitostanol, stigmasterol, stigmastanol, campesterol, brassicasterol), ergosterol, retinol, cholecalciferol, ergocalciferol, tocopherol, or tocotrienol.

Another embodiment of the third aspect of the disclosure is a PALM-cargo molecule complex wherein the cargo molecule is a compound conjugate of formula I: wherein A is an agent having a hydroxy or amine group; R is the hydroxyl or the amine group of the agent; L is selected from the group consisting of carbonic acid, succinic acid or diglycolic acid; and X is selected from the group consisting of cholesterol, α-tocotrienol, β-tocotrienol, γ-tocotrienol, δ-tocotrienol, cholesterol, coprostanol, plant sterols, (ß-sitosterol, sitostanol, stigmasterol, stigmastanol, campesterol, brassicasterol), ergosterol, retinol, cholecalciferol, ergocalciferol, α-tocopherol, β-tocopherol, γ-tocopherol, and δ-tocopherol,

Another embodiment of the third aspect of the disclosure is a PALM-cargo molecule complex wherein the cargo molecule is a compound conjugate of formula I: wherein A is an agent having a hydroxy or amine group; R is a hydroxyl or an amine group of the agent; L is a linker; and X is an anchor moiety selected from the group consisting of cholesterol, cholecalciferol and δ-tocotrienol.

In one embodiment of a compound conjugate of formula (1), R is a hydroxy group of the agent, and the anchor moiety is covalently bonded to agent by a carbonate ester bond. In another embodiment of a compound conjugate of formula (1), R is an amine group of the agent, and the anchor moiety is covalently bonded to agent by a carbamate ester bond.

In another embodiment of a compound conjugate of formula (1), the anchor moiety is cholesterol. In still another embodiment of a compound conjugate of formula (1), the anchor moiety is cholesterol, with the proviso that if the anchor moiety is cholesterol, then the compound is not paclitaxel.

In yet another embodiment of a compound conjugate of formula (1) the anchor moiety is α-tocotrienol. In another embodiment of a compound conjugate of formula (1) the anchor moiety is β-tocotrienol. In still another embodiment of a compound conjugate of formula (1) the anchor moiety is γ-tocotrienol. In yet another embodiment of a compound conjugate of formula (1) the anchor moiety is δ-tocotrienol. In still another embodiment of a compound conjugate of formula (1) the anchor moiety is ergocalciferol.

In some embodiments of the compound conjugate of formula (1) the agent is a drug.

In some embodiments of the compound conjugate of formula (1) the agent is a chemotherapeutic agent that causes and/or is associated with CIPN. In one embodiment of the compound conjugate of formula (1) the agent is a CIPN causing chemotherapeutic agent and the chemotherapeutic agent is covalently bonded to the anchor by a carbonate ester bond.

In one embodiment of the compound conjugate of formula (1) the agent is a CIPN causing chemotherapeutic agent and the CIPN causing chemotherapeutic agent is covalently bonded to the anchor by a carbamate ester bond.

Non-limiting examples of a CIPN causing chemotherapeutic agents having a hydroxyl group available to form the carbonate ester bond include vincristine, des-acetyl vinblastine, des-acetyl vinorelbine, tubulysin A, epothilone B, ixabepilone, eribulin, emtansine, docetaxel, cabazitaxel, or paclitaxel.

Non-limiting examples of CIPN causing chemotherapeutic agents having an amine available for forming the carbamate ester bond include, gemcitabine and cytarabine.

In some embodiments of the PALM-chemotherapeutic agent conjugated nanoparticle compositions of the third aspect of the disclosure, the CIPN causing chemotherapeutic agent is paclitaxel 2′-cholesteryl carbonate. In another embodiment the chemotherapeutic agent is paclitaxel 2′-δ-tocotrienyl carbonate.

In yet other embodiments, the CIPN causing chemotherapeutic agent is docetaxel 2′-cholesteryl carbonate. In other embodiments, the CIPN causing chemotherapeutic agent is the cholesteryl carbonate ester of gemcitabine. In other embodiments, the CIPN causing chemotherapeutic agent is the cholesteryl carbonate ester of tubulysin A.

In other embodiments of the PALM-chemotherapeutic agent conjugated nanoparticle compositions of the third aspect of the disclosure, the CIPN causing chemotherapeutic agent is the cholesteryl carbamate ester of gemcitabine (Cholesteryl (N⁴)-Gemcitabine Carbamate).

In yet another embodiments, the CIPN causing chemotherapeutic agent is the cholesteryl carbonate ester of vincristine, the structure of which is:

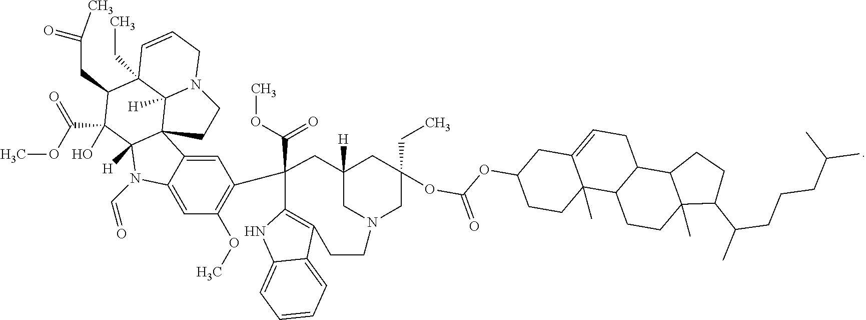

In still another embodiment the CIPN causing chemotherapeutic agent is the delta-tocotrienyl carbamate ester of paclitaxel, the structure of which is:

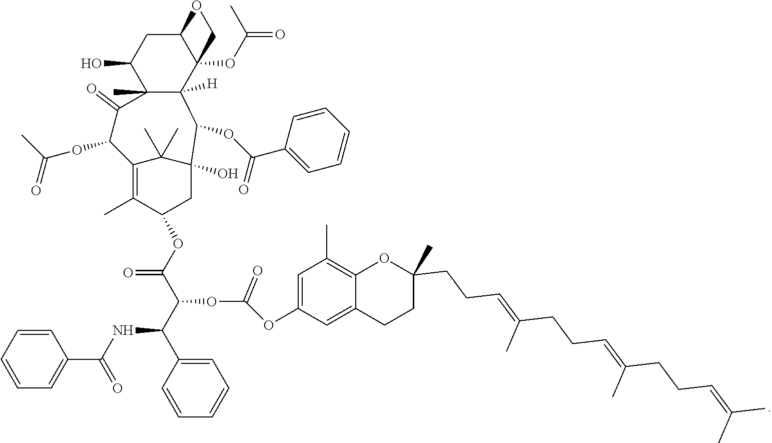 [SMILES: CC(=O)O[C@@H]1C(=O)[C@@]2(C)[C@H]([C@H](OC(=O)c3ccccc3)[C@@]3(O)C[C@H](OC(=O)[C@H](OC(=O)Oc4cc(C)c5c(c4)CC[C@@](C)(CC/C=C(\C)CC/C=C(\C)CCC=C(C)C)O5)[C@H](NC(=O)c4ccccc4)c4ccccc4)C(C)=C1C3(C)C)[C@]1(OC(C)=O)CO[C@@H]1C[C@@H]2O]

In still another embodiment the CIPN causing chemotherapeutic agent is the gemcitabine delta-tocotrienlyl carbonate ester, the structure of which is:

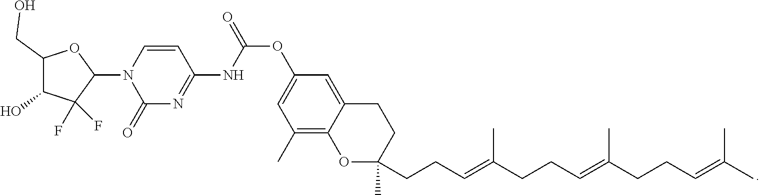 [SMILES: CC(C)=CCC/C(C)=C/CC/C(C)=C/CC[C@]1(C)CCc2cc(OC(=O)Nc3ccn(C4OC(CO)[C@@H](O)C4(F)F)c(=O)n3)cc(C)c2O1]

Table 4 provides the structure of non-limiting examples of CIPN causing chemotherapeutic agent (A) useful in the present invention with the hydroxyl or amine group (R) indicated by an arrow.

TABLE 4

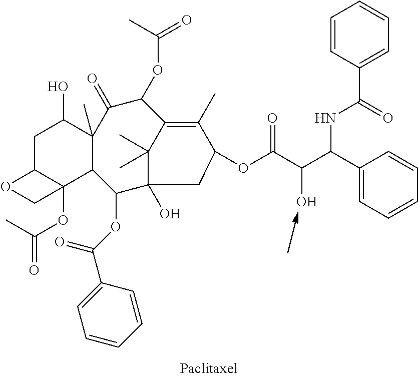 [SMILES: CC(=O)OC1C(=O)C2(C)C(O)CC3OCC3(OC(C)=O)C2C(OC(=O)c2ccccc2)C2(O)CC(OC(=O)C(O)C(NC(=O)c3ccccc3)c3ccccc3)C(C)=C1C2(C)C]

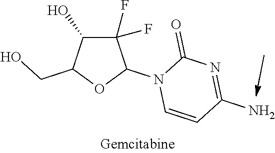 [SMILES: Nc1ccn(C2OC(CO)[C@H](O)C2(F)F)c(=O)n1]

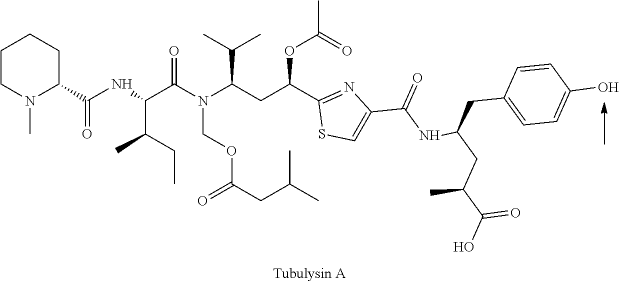 [SMILES: CC[C@@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C]

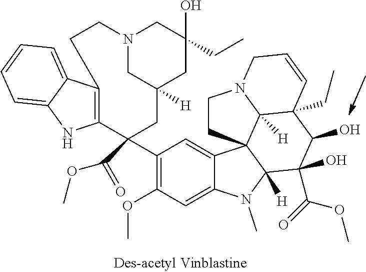

Table 5 provides non-limiting examples of PALM-chemotherapeutic agent compositions of formula A—R—L—X.

TABLE 5

| Compound | A | R | L | X |
|---|---|---|---|---|
| 1 | Paclitaxel | OH | Carbonic Acid | γ-Tocotrienol |
| 2 | Paclitaxel | OH | Carbonic Acid | δ-Tocotrienol |
| 3 | Paclitaxel | OH | Carbonic Acid | Cholecalciferol |
| 4 | Paclitaxel | OH | Carbonic Acid | Ergocalciferol |
| 5 | Paclitaxel | OH | Succinic Acid | Cholesterol |
| 6 | Paclitaxel | OH | Succinic Acid | γ-Tocotrienol |
| 7 | Paclitaxel | OH | Succinic Acid | δ-Tocotrienol |
| 8 | Paclitaxel | OH | Succinic Acid | Cholecalciferol |
| 9 | Paclitaxel | OH | Succinic Acid | Ergocalciferol |
| 10 | Paclitaxel | OH | Diglycolic Acid | Cholesterol |
| 11 | Paclitaxel | OH | Diglycolic Acid | γ-Tocotrienol |
| 12 | Paclitaxel | OH | Diglycolic Acid | δ-Tocotrienol |
| 13 | Paclitaxel | OH | Diglycolic Acid | Cholecalciferol |
| 14 | Paclitaxel | OH | Diglycolic Acid | Ergocalciferol |
| 15 | Gemcitabine | NH₂ | Carbonic Acid | Cholesterol |
| 16 | Gemcitabine | NH₂ | Carbonic Acid | γ-Tocotrienol |
| 17 | Gemcitabine | NH₂ | Carbonic Acid | δ-Tocotrienol |
| 18 | Gemcitabine | NH₂ | Carbonic Acid | Cholecalciferol |
| 19 | Gemcitabine | NH₂ | Carbonic Acid | Ergocalciferol |
| 20 | Gemcitabine | NH₂ | Succinic Acid | Cholesterol |
| 21 | Gemcitabine | NH₂ | Succinic Acid | γ-Tocotrienol |
| 22 | Gemcitabine | NH₂ | Succinic Acid | δ-Tocotrienol |
| 23 | Gemcitabine | NH₂ | Succinic Acid | Cholecalciferol |
| 24 | Gemcitabine | NH₂ | Succinic Acid | Ergocalciferol |
| 25 | Gemcitabine | NH₂ | Diglycolic Acid | Cholesterol |
| 26 | Gemcitabine | NH₂ | Diglycolic Acid | γ-Tocotrienol |
| 27 | Gemcitabine | NH₂ | Diglycolic Acid | δ-Tocotrienol |
| 28 | Gemcitabine | NH₂ | Diglycolic Acid | Cholecalciferol |
| 29 | Gemcitabine | NH₂ | Diglycolic Acid | Ergocalciferol |
| 30 | Tubulysin A | OH | Carbonic Acid | Cholesterol |
| 31 | Tubulysin A | OH | Carbonic Acid | γ-Tocotrienol |
| 32 | Tubulysin A | OH | Carbonic Acid | δ-Tocotrienol |
| 33 | Tubulysin A | OH | Carbonic Acid | Cholecalciferol |
| 34 | Tubulysin A | OH | Carbonic Acid | Ergocalciferol |
| 35 | Tubulysin A | OH | Succinic Acid | Cholesterol |
| 36 | Tubulysin A | OH | Succinic Acid | γ-Tocotrienol |
| 37 | Tubulysin A | OH | Succinic Acid | δ-Tocotrienol |
| 38 | Tubulysin A | OH | Succinic Acid | Cholecalciferol |
| 39 | Tubulysin A | OH | Succinic Acid | Ergocalciferol |
| 40 | Tubulysin A | OH | Diglycolic Acid | Cholesterol |
| 41 | Tubulysin A | OH | Diglycolic Acid | γ-Tocotrienol |
| 42 | Tubulysin A | OH | Diglycolic Acid | δ-Tocotrienol |
| 43 | Tubulysin A | OH | Diglycolic Acid | Cholecalciferol |
| 44 | Tubulysin A | OH | Diglycolic Acid | Ergocalciferol |

A fourth aspect of the disclosure provides for a surprisingly effective co-lyophilization techniques to produce PALM or PALM-chemotherapeutic agent nanoparticle compositions from a homogenous solvent phase composed of tert-butyl alcohol and water. The advantages of this approach are: 1) all PALM constituents including peptide, phosphopholipid and optional lipophilic cargo (e.g. a CIPN causing chemotherapeutic agent), for example, paclitaxel-2′-cholesteryl carbonate, are co-solubilized in a single solvent phase, 2) the solvent components are totally miscible and well-suited to removal by standard lyophilization procedure, 3) the procedures avoids potentially toxic substances because tert-butyl alcohol is a low toxicity, class 3 solvent and 4) the resultant dried lyophilizate enables opportunities for greater stability during storage than is possible with aqueous preparations.

The solvent mixture used to prepare PALM is preferably a mixture of tert-butyl alcohol (TBA) and water. In one embodiment the percent ration of TBA to water is between about 70%:30% to about 90%:10%. In another embodiment the ratio is between about 75%:25% and about 85%:15%. In yet another embodiment the ratio is 80%:20%.

One embodiment of the fourth aspect provides a process for preparing PALM comprises the steps:
i) solubilizing an amphiphilic peptide in a first solvent mixture to provide a peptide solution;
ii) solubilizing a sphingomyelin in a second solvent mixture to provide a sphingomyelin solution
iii) solubilizing an additional phospholipid in a third solvent mixture to provide a phospholipid solution;
iv) combining the peptide solution, the sphingomyelin solution and the phospholipid solution to form a peptide/sphingomyelin/phospholipid solution; and
v) lyophilizing the peptide/sphingomyelin/phospholipid solution,
   wherein steps i), ii), and iii) are performed in any order; and wherein the first, second, and third solvent mixture comprises tert-butyl alcohol and water.

Another embodiment of the fourth aspect of the disclosure provides a process for preparing PALM comprises the steps:
i) combining an amphiphilic peptide, sphingomyelin and an additional phospholipid, to form a peptide/sphingomyelin/phospholipid mixture;
ii) solubilizing the peptide/sphingomyelin/phospholipid mixture in a solvent mixture to form a peptide sphingomyelin/phospholipid solution; and
iii) lyophilizing the peptide/phospholipid solution,
   wherein the solvent mixture comprises tert-butyl alcohol and water.

The fourth aspect of the present disclosure additionally provides a process for preparing PALM comprising a CIPN causing chemotherapeutic agent to form a PALM-chemotherapeutic agent nanoparticle. To prepare a PALM-chemotherapeutic agent nanoparticle, the peptide, sphingomyelin, one or more additional phospholipid and a CIPN causing chemotherapeutic agent are each separately prepared in a solvent mixture and, depending on the desired formulation, are combined in specific molar ratios. Alternately, the peptide, sphingomyelin, one or more additional phospholipid and a CIPN causing chemotherapeutic agent can be combined directly, without prior solubilization, and then brought into solution with the desired solvent mixture prior to lyophilization.

One embodiment of the fourth aspect of the disclosure provides a process for preparing a PALM-chemotherapeutic agent nanoparticle comprising the steps:
i) solubilizing an amphiphilic peptide in a first solvent mixture to provide a peptide solution;
ii) solubilizing a sphingomyelin in a second solvent mixture to provide a sphingomyelin solution
iii) solubilizing an additional phospholipid in a third solvent mixture to provide a phospholipid solution;
iv) solubilizing a CIPN causing chemotherapeutic agent in a fourth solvent mixture to provide a cargo molecule solution;
v) combining the peptide solution, the sphingomyelin solution, the phospholipid solution and the CIPN causing chemotherapeutic agent solution to form a peptide/sphingomyelin/phospholipid/chemotherapeutic agent solution; and
vi) lyophilizing the peptide/sphingomyelin/phospholipid/chemotherapeutic agent solution,
   wherein steps i) ii), iii) and iv) are performed in any order; and wherein the first, second, third and fourth solvent mixture comprise tert-butyl alcohol and water.

Another embodiment of preparing a PALM-chemotherapeutic agent nanoparticle comprises the steps:
i) combining an amphiphilic peptide, sphingomyelin, an additional phospholipid and a cargo molecule, to form a peptide/sphingomyelin/phospholipid/cargo molecule mixture;
ii) solubilizing the peptide/sphingomyelin/phospholipid/cargo molecule mixture in a solvent mixture to form a peptide/phospholipid solution; and
iii) lyophilizing the peptide/sphingomyelin/phospholipid/chemotherapeutic agent solution,
   wherein the solvent mixture comprises tert-butyl alcohol and water

The resultant lyophilized cake can be stored for long periods of time and will remain stable. The lyophilized product is rehydrated by adding any suitable aqueous solution, e.g., water or saline, followed by gentle swirling of the contents. Reconstitution of PALM lyophilizates can be enhanced by incubation of the PALM solution at 50° C. for from 5 to 30 minutes. The solution is then filter sterilized (0.2 μm) and stored at 4-8° C. Alternately, the solvent mixture comprising the peptide, phospholipid and the cargo molecule is filter sterilized prior to lyophilization.

A fifth aspect of the present disclosure provides methods for treating CIPN, for example, PIPN, (paclitaxel induced peripheral neuropathy) comprising administering to a subject in need thereof, an effective amount of a composition comprising a PALM-chemotherapeutic agent containing nanoparticle composition according to any one the embodiments of the disclosure.

Scavenger receptor B-1 (SR-B1) is a membrane receptor that binds apolipoprotein A-I, the principle protein component of HDL, to facilitate cellular transport of cholesterol. Cholesterol is an essential nutrient for proliferating cells like those found in malignant tumors. SR-B1 is highly expressed in many tumor cells, including but not limited to breast, prostate, colorectal, pancreatic, adrenal, skin, nasopharyngeal and ovarian cancers. Some amphiphilic peptides are also recognized and bound by SR-BI. PALM are formed from combinations of phospholipid and amphiphilic peptides designed to bind to SR-BI and thereby to selectively deliver chemotherapeutic agent to SR-BI-positive cells.

For pharmaceutical use, lyophilized PALM may be provided in single dose or multiple dose containers that can be conveniently reconstituted at the point of use, e.g., hospital or doctor's office using standard diluents such as sterile water for injection, normal sterile saline or sterile 5% dextrose solution. Suitable containers are then aseptically filled with the sterilized mixture, lyophilized and sealed appropriately to maintain sterility of the lyophilized material. Suitable containers include but are not limited to a vial comprising a rubber seal, or the equivalent, that allows for introduction of a diluent for reconstitution, e.g., via a syringe. Such PALM preparations are suitable for parenteral administration including intravenous, subcutaneous, intramuscular, intraperitoneal injection.

This invention also is directed, in part, to all compositions comprising a PALM and a CIPN causing chemotherapeutic agent, and methods of their use. PALM molecules and their CIPN causing chemotherapeutic agent(s) may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Total daily dose of the PALM of the invention to be administered to a human or other mammal host in single or divided doses may be in amounts, for example, from 0.1 to 300 mg/kg body weight daily and more usually 0.1 to 200 mg/kg body weight daily, or the dose, from 0.1 to 100 mg/kg body weight daily.

In one embodiment of the invention, the dose of the PALM-chemotherapeutic agent nanoparticles (in total), is in the range of 0.1 to 300 mg/kg, the range of 5 to 200 mg/kg, the range of 10 to 100 mg/kg, or the range of 10 mg/kg to 50 mg/kg. In a further embodiment of the invention, the dose of PALM molecules and the chemotherapeutic agent (in total), is about 5 mg/kg, 10 mg/kg, 20 mg/kg, 40 mg/kg, 50 mg/kg, 60 mg/kg, 70 mg/kg, 80 mg/kg, 90 mg/kg, or 100 mg/kg, 200 mg/kg, 300 mg/kg. The dose can be administered once a day. The dose can be administered three times a week. Alternatively, the dose can be administered twice a week. Alternatively, the dose can be administered once a week. In another embodiment the dose can be administered once a month.

In related embodiments, the amount of chemotherapeutic agent in the combination of PALM and chemotherapeutic agent, the dose may range from about 0.01 mg to about 35 mg per kilogram body weight, about 0.01 mg to about 30 mg per kilogram body weight, about 0.01 mg to about 25 mg per kilogram body weight, about 0.01 to about 20 mg per kilogram body weight, or about 0.01 to about 10 mg per kilogram body weight, of the patient. In further related embodiments, the amount of chemotherapeutic agent in the composition with the PALM molecules is a prescribed Food and Drug Administration (FDA USA) or European Medicines Agency (EMA) approved dose of chemotherapeutic for the treatment of cancer the patient may have or is treated with.

In one embodiment, the CIPN is sensory. In one embodiment, the neuropathy presents as distal axonopathy. In another embodiment, the neuropathy presents as dysesthesia, paraesthesia, burning, numbness, and/or pain.

In one embodiment, the CIPN is motor. In another embodiment, the neuropathy presents as myoatrophy. In another embodiment, the neuropathy presents with loss of distal deep tendon reflexes.

In one embodiment, CIPN is autonomic.

In one embodiment, the subject has an elevated risk of developing chemotherapy-induced peripheral neuropathy. Subjects with an elevated risk of developing CIPN have preexisting conditions including diabetes, nutritional deficiency, alcoholism, and previous exposure to neurotoxic chemotherapy. In another embodiment, the subject has a past history of neuropathy. The previous neuropathy may have been caused by diabetes, nutritional deficiency, alcoholism, hereditary disease and/or neurotoxic chemotherapy.

In one embodiment, the present invention further comprises the step of administering one or more chemotherapeutic agents, in addition to the chemotherapeutic agent accompanying the PALM containing composition.

In various embodiments, the chemotherapeutic agent or agents in the PALM containing composition and/or cargo molecule, may include, for example, antimetabolites (i.e., folate antagonists, purine antagonists, and pyrimidine antagonists), bleomycins, DNA alkylating agents (i.e., nitrosoureas, cross linking agents, and alkyating agents), hormones, aromatase inhibitors, monoclonal antibodies, antibiotics, platinum complexes, protesome inhibitors, taxane analogs, vinca alkaloids, topoisomerase inhibitors (i.e., anthracyclines, camptothecins, podophyllotoxins), tyrosine kinase inhibitors, or a combination thereof.

In another embodiment, the chemotherapeutic agent or agents in the PALM containing composition and/or cargo molecule, may include, for example, a platinum complex, a vinca analog, a taxane analog, an alkylating agent, an antimetabolite, a proteasome inhibitor, or a combination thereof.

Platinum complexes may include, for example, cisplatin, oxaliplatin, eptaplatin, lobaplatin, nedaplatin, carboplatin, satraplatin, picoplatin, miriplatin and the like.

Vinca alkaloids may include, for example, vincristine, vinblastine, vinorelbine, vindesine, and the like.

Taxanes may include, for example, paclitaxel, docetaxel, cabazitaxel and various formulations and analogs thereof.

Alkylating agents may include, for example, dacarbazine, procarbazine, temozolamide, thiotepa, mechlorethamine, chlorambucil, L-phenylalanine mustard, melphalan, ifosphamide, cyclophosphamide, mefosphamide, perfosfamide, trophosphamide, busulfan, carmustine, lomustine, thiotepa, semustine, and the like.

Antimetabolites include pemetrexed disodium, 5 azacitidine, capecitabine, carmofur, cladribine, clofarabine, cytarabine, cytarabine ocfosfate, cytosine arabinoside, decitabine, deferoxamine, doxifluridine, eflornithine, enocitabine, ethnylcytidine, fludarabine, 5 fluorouracil alone or in combination with leucovorin, gemcitabine, hydroxyurea, melphalan, mercaptopurine, 6 mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Proteasome inhibitors may include, for example, bortezomib.

Topoisomerase inhibitors include aclarubicin, 9-aminocamptothecin, amonafide, amsacrine, becatecarin, belotecan, irinotecan hydrochloride, camptothecin, dexrazoxine, diflomotecan, edotecarin, epirubicin, etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

In another embodiment, chemotherapeutic agents are bortezomib, carboplatin, cisplatin, misonidazole, oxaliplatin, procarbazine, thalidomide, docetaxel, hexamethylmelamine, paclitaxel, vincristine, vinblastine, or vinorelbine.

In one embodiment, the chemotherapeutic agent is docetaxel, paclitaxel, carboplatin, doxorubicin, cisplatin, oxaliplatin, capecitabine, 5-fluorouracil and leucovorin.

In various embodiments, the patient experiencing CIPN, or is likely to experience CIPN is undergoing or has previously been treated with a chemotherapeutic agent, for example, one or more of docetaxel, paclitaxel, carboplatin, cisplatin, gemcitabine, oxaliplatin, capecitabine, 5-fluorouracil and leucovorin that causes CIPN in the patient thus treated, or is associated with CIPN in the patient or likely to cause CIPN in the patient. In these patients, the method of treatment or prevention of CIPN, for example PIPN, includes providing a chemotherapeutic for which the patient is or was being treated with that is associated in a new formulation, that formulation comprising a composition containing PALM, to treat the patient's CIPN or to prevent the occurrence of CIPN in the treated cancer patient.

In another embodiment, the chemotherapeutic agent or agents is administered for the treatment of cancer.

In one embodiment of the invention, the cancer being treated is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In yet another embodiment of the invention, the cancer being treated is selected from the group consisting of ovarian cancer, cervical cancer, colorectal cancer, prostate cancer, breast cancer, gastric adenocarcinoma, head and neck cancer, testicular cancer, leukemia, neuroblastoma, Hodgkin's lymphoma, non-Hodgkin's lymphoma, and non-small cell lung cancer.

The administration of a composition comprising a PALM and a chemotherapeutic agent, and formulations thereof, may be prior to, immediately prior to, during, immediately subsequent to or subsequent to the administration of the one or more chemotherapeutic agents. The composition comprising a PALM and a chemotherapeutic agent, can be administered prophylactically before CIPN is established or for treating established CIPN. The established CIPN can be acute or chronic.

In various embodiments of the methods of prevention and treatment of CIPN contemplated herein, the compositions administered to a cancer patient pre, during or post CIPN, may contain an amount of the chemotherapeutic agent ranging from about 5 mg to about 5,000 mg, which may comprise an effective dose, or a sub-effective dose, or a daily dose, or a divided daily dose, of said chemotherapeutic agent.

In some embodiments, cisplatin can be administered at a range of 20 mg/m² to 140 mg/m² in cycles of 1, 2, 3, 4, 5, 6, 7, or 8. For example, cisplatin can be administered at 20 mg/m² daily for five days per cycle. Cisplatin can be administered at 75 to 100 mg/m² once per cycle every four weeks (Day 1). Cisplatin can be administered 50 to 70 mg/m² once per cycle every three to four weeks (Day 1).

Carboplatin can be administered at about 300 mg/m² or less or at about 360 mg/m² or less once per cycle every three to four weeks (Day 1). Carboplatin can be administered in cycles of 1, 2, 3, 4, 5, 6, 7, or 8.

Oxaliplatin can be administered at about 85 mg/m² or less once per cycle every 2 weeks. Oxaliplatin can be administered in cycles of 1, 2, 3, 4, 5, 6, 7, or 8.

Docetaxel can be administered at about 60 mg/m² to about 100 mg/m² in cycles of 1, 2, 3, 4, 5, 6, 7, or 8. For example, docetaxel can be administered at 75 mg/m² once per cycle every three weeks (Day 1).

Paclitaxel can be administered at a range of about 100 mg/m² to about 175 mg/m² in cycles of 1, 2, 3, 4, 5, 6, 7, or 8. Paclitaxel can be administered at about 100 mg/m² once per cycle every 3 weeks (Day 1). Paclitaxel can be administered at about 135 mg/m² once per cycle every 3 weeks (Day 1). Paclitaxel can be administered at about 175 mg/m² once per cycle every 3 weeks (Day 1).

Vincristine can be administered at a range of about 0.4 mg/m² to 1.4 mg/m² once per cycle every one to four weeks (Day 1). Vincrinstine can be administered in cycles of 1, 2, 3, 4, 5, 6, 7, or 8.

Vinblastine can be administered at a range of about 3.7 mg/m² to about 18.5 mg/m² once per cycle every one to four weeks (Day 1). For example, vinblastine can be administered at 3.7 mg/m², 5.5 mg/m², 7.4 mg/m², 9.25 mg/m², or 11.1 mg/m². Vinblastine can be administered in cycles of 1, 2, 3, 4, 5, 6, 7, or 8.

Vinorelbine can be administered at a range of about 25 m g/m.sup.² to about 120 mg/m² once per cycle every one to six weeks (Day 1). For example, vinorelbine can be administered at 30 mg/m². Vinorelbine can be administered in cycles of 1, 2, 3, 4, 5, 6, 7, or 8.

In one embodiment, compositions comprising PALM and a chemotherapeutic agent and formulations thereof are administered once a day during the treatment cycle e.g. is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent formulations thereof are administered twice a day during the treatment cycle e.g. is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent and formulations thereof are administered twice a week during the treatment cycle e.g. is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent and formulations thereof are administered once a week during the treatment cycle e.g. is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent and formulations thereof are administered once a week during the treatment cycle e.g. is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent and formulations thereof are administered once a day during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent formulations thereof are administered twice a day during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent and formulations thereof are administered twice a week during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent and formulations thereof are administered once a week during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In one embodiment, compositions comprising PALM and a chemotherapeutic agent and formulations thereof are administered once a week during the treatment cycle wherein a chemotherapeutic agent or agent(s) is administered at Day 1 of the cycle, wherein a cycle is 5 days, 1 week, 2 weeks, 3 weeks, 4 weeks, 5 weeks or 6 weeks.

In another embodiment, compositions comprising PALM and a chemotherapeutic agent and formulations thereof are administered at least one day prior to chemotherapy. In another embodiment, compositions comprising PALM and a chemotherapeutic agent and formulations thereof are administered for two days prior to chemotherapy. In another embodiment, compositions comprising PALM and a chemotherapeutic agent and formulations thereof are administered for one week prior to chemotherapy. In yet another embodiment, compositions comprising PALM and a chemotherapeutic agent and formulations thereof are administered immediately prior to each chemotherapy treatment. In yet another embodiment, compositions comprising PALM and a chemotherapeutic agent and formulations thereof are administered simultaneously with each chemotherapy treatment. In yet another embodiment, compositions comprising PALM and a chemotherapeutic agent and formulations thereof are administered subsequent to chemotherapy.

In a subset of the above embodiments, chemotherapy and chemotherapy treatment may include single administration or multiple administrations of the compositions of the present disclosure, e.g. compositions comprising PALM and a chemotherapeutic agent in the absence of any additional chemotherapeutic agents. In other related embodiments as exemplified above, the chemotherapy and chemotherapy treatment comprises administration of a different chemotherapeutic agent to the chemotherapeutic agent present in the compositions and formulations comprising PALM and a chemotherapeutic agent, and reference to chemotherapy and chemotherapy treatment means administration of a different chemotherapeutic agent as the chemotherapeutic agent present in the composition containing PALM and a chemotherapeutic agent.

The invention further allows for administration of higher dose of chemotherapy. Additionally, the invention allows for administration of additional cycles of chemotherapy. The invention also allows for reduction of time between cycles of chemotherapy.

The severity of the incidence of CIPN is reflected in the grade, i.e., 0, 1, 2, 3, or 4. The scale escalates from grade 0), normal and asymptomatic, to grade 4, disabling and/or life-threatening. (Postma T. J., Annals of Oncology 1998 9:739-744). Grade 3 requires corrective measures, including dose reduction and/or delays.

There are multiple Common Toxicity Criteria (CTC) scales used in clinical practice to evaluate the severity of CIPN; World Health Organization (WHO) scale, Eastern Cooperative Oncology Group (ECOG) scale, National Cancer Institute-Common Toxicity Criteria (NCI-CTC), and Ajani scale. (Cavaletti G., et al., European Journal of Cancer 2010) 46:479-494). The scales represent a combination of objective assessment and the patients' perception of CIPN effects.

One embodiment of the invention provides methods of treating, including treating prophylactically, chemotherapy-induced peripheral neuropathy with a composition of the present invention containing PALM and a chemotherapeutic agent, wherein the incidence of grade 3 or 4 CIPN is decreased. In another embodiment, the incidence of grade 1 or 2 CIPN is decreased. In another embodiment, the incidence of grade 3 or 4 CIPN is decreased to grade 1 or 2 CIPN. In another embodiment, the incidence of grade 2 CIPN is decreased to grade 1.

The present invention further provides a method for mitigating neurotoxic effects of a chemotherapeutic agent, wherein incidence of grade 3 or 4 CIPN is decreased. In another embodiment, the incidence of grade 1 or 2 CIPN is decreased. In another embodiment, the incidence of grade 3 or 4 CIPN is decreased to grade 1 or 2 CIPN. In another embodiment, the incidence of grade 2 CIPN is decreased to grade 1.

Alternatively, CIPN can be evaluated with a quality of life assessment. One such assessment is the European Organization of Research and Treatment of Cancer (EORTC) QLQ-CIPN20) questionnaire. (Cavaletti G., et al., European Journal of Cancer 2010 46:479-494).

In one embodiment of the invention, CIPN is improved on EORTC QLQ-CIPN 20 questionnaire, when the cancer patient is administered one or more administrations of the compositions of the present invention containing PALM and a chemotherapeutic agent, wherein the agent causing or associated with the CIPN is the same chemotherapeutic agent present in the compositions of the present invention.

One embodiment of the invention provides methods of treating or preventing chemotherapy-induced neuropathic pain with a compositions of the present invention containing PALM and a chemotherapeutic agent. Neuropathic pain is the intractable pain caused by dysfunction in the peripheral or central nervous system.

Pain can be evaluated with a quality of life assessment. One such assessment is the European Organization of Research and Treatment of Cancer (EORTC) EORTC QLQ-C30/L13 questionnaire.

In one embodiment of the invention, the pain is decreased based on the assessment of the EORTC QLQ-C30/L13 questionnaire.

In one embodiment of the invention, the pain is peripheral neuropathic pain or central neuropathic pain.

In another embodiment of the invention, the pain is chronic or acute.

In another embodiment of the invention, the use of supportive care for pain is reduced. Supportive care includes, for example, NSAIDS or opioids.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1. Peptide Synthesis and Purification

Peptides were produced by standard Fmoc solid-phase synthesis techniques at GenScript USA, Inc. (Piscataway, NJ). Certain peptides were modified at the terminal amino acids by acetylation of the N-terminus and amidation of the C-terminus by standard procedures. Peptides were chromatographically purified to greater than 95% purity by a standard high-performance liquid chromatography method for peptide purification. Purity was confirmed by HPLC and mass spectroscopic analysis.

### Example 2. Paclitaxel 2′-Cholesteryl Carbonate (XC) Synthesis

Fifty milligrams of paclitaxel was dissolved in 2 ml of chloroform and then combined with 1.5 molar excess of cholesterol chloroformate in 2 ml of chloroform plus 4 ml of N,N-diisopropylethylamine and 2 ml acetonitrile. The mixture was stirred overnight at ambient temperature and then dried on a rotary evaporator. The resulting off-white precipitate was then dissolved in ethyl acetate/hexane (3:1) and extracted with water, dried, and then redissolved in chloroform. The formation of the product was confirmed by thin-layer chromatography using ethyl acetate/hexane (3:1) as the mobile phase (Rf of paclitaxel 0.4, Rf of Tax-Chol 0.92). Further purification of the product was then carried out on a silica gel column using ethyl acetate/hexane (3:1) as the mobile phase to yield the titled compound (1). The structure was confirmed by mass spectrometry and NMR analysis.

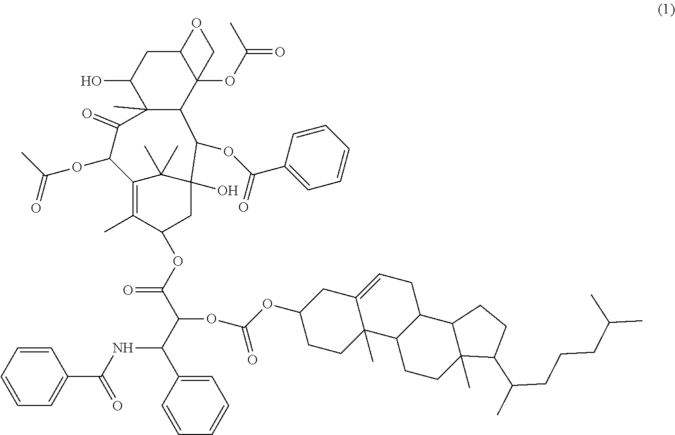 [SMILES: CC(=O)OC1C(=O)C2(C)C(O)CC3OCC3(OC(C)=O)C2C(OC(=O)c2ccccc2)C2(O)CC(OC(=O)C(OC(=O)OC3CCC4(C)C(=CCC5C4CCC4(C)C(C(C)CCCC(C)C)CCC54)C3)C(NC(=O)c3ccccc3)c3ccccc3)C(C)=C1C2(C)C]

### Example 3. Paclitaxel 2′-δ-Tocotrienyl Carbonate (XTT or Also Known as XT3 or Compound 1) Synthesis

Step 1. Synthesis of P-Nitrophenyl Carbonate pf Delta-Tocotrienol

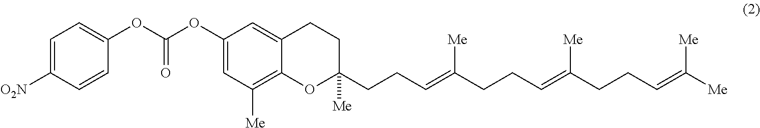 [SMILES: CC(C)=CCC/C(C)=C/CC/C(C)=C/CC[C@]1(C)CCc2cc(OC(=O)Oc3ccc([N+](=O)[O-])cc3)cc(C)c2O1]

To the solution of delta-tocotrienol (25 Mg, 0.0629 mmol) in anhydrous methylene chloride (1.5 mL), was added 4-nitrophenyl chloroformate (51 Mg, 0.25 mmol) and triethylmine (35 μL, 0.25 mmol) at room temperature. The reaction mixture was stirred at room temperature for 24 h and then concentrated and then the desired product (2) was obtained using preparative TLC using ethyl acetate/heptanes (10:90) as eluent. The desired product was obtained as yellow powder (18 Mg). ¹H NMR (CDCl₃): δ8.30 (d, 2H), 7.45 (d, 2H), 6.80 (dd, 2H), 5.05-5.20 (m, 3H), 2.72-2.78 (t, 2H), 2.18 (s, 4H), 1.95-2.15 (m, 4H), 1.72-1.85 (m, 4H), 1.68 (s, 3H), 1.55-1.62 (br s, 12 H), 1.30 (br s, 5H).

Step 2. Synthesis of Delta-Tocotrienol Carbonate of Paclitaxel (3)

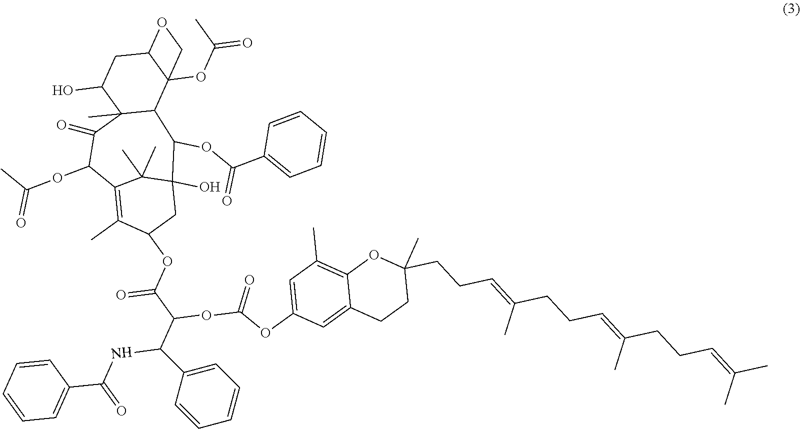 [SMILES: CC(=O)OC1C(=O)C2(C)C(O)CC3OCC3(OC(C)=O)C2C(OC(=O)c2ccccc2)C2(O)CC(OC(=O)C(OC(=O)Oc3cc(C)c4c(c3)CCC(C)(CC/C=C(\C)CC/C=C(\C)CCC=C(C)C)O4)C(NC(=O)c3ccccc3)c3ccccc3)C(C)=C1C2(C)C]

A solution of compound (2) (18 mg, step 1 product) in methylene chloride (2 mL), paclitaxel (28 mg) and DMAP (10 Mg) are combined at room temperature. The mixture was stirred at room temperature for 24 h. The mixture was concentrated and purified by using preparative TLC using ethyl acetate/heptanes (50:50) as eluent. The desired product (3) (17 Mg) was obtained as colorless solid. TLC Analysis (Rf 0.25, EA/Hexanes: 1:1). ¹H NMR (CDCl₃): δ8.20 (d, 2H), 7.75 (d, 2H), 7.60-7.62 (m, 1H), 7.30-7.52 (m, 9H), 6.90-6.95 (d, 1H), 6.60-6.75 (dd, 2H), 6.20-6.30 (m, 2H), 6.00-6.05 (m, 1H), 5.70-5.75 (d, 1H), 5.50 (s, 1H), 5.10-5.20 (br s, 2H), 4.95-5.00 (d, 1H), 4.30-4.35 (br s, 1H), 4.20-4.30 (dd, 2H), 3.75-3.80 (d, 1H), 2.70-2.75 (m, 2H), 2.30-2.60 (m, 7H), 2.23-2.27 (m, 11H), 1.50-2.20 (m, 26H), 1.25 (m, 9H), 1.15 (s, 3H)

### Example 4. Peptide Amphiphile Lipid Micelle (PALM) Preparation

Separate stock solutions of peptide and phospholipids were prepared in a solvent mixture composed of 80% tert-butyl alcohol (TBA) and 20% water to obtain separate solutions of 10 mM peptide, 20 mM 1,2-dioleoyl-sn-glycero-3-phosphocholine (DOPC) or 20 mM 1-palmitoyl-2-oleoyl-phosphatidylcholine (POPC) and 20 mM egg SM. Aliquots of stock solution were combined to obtain a final solution containing 10 mole equivalents of peptide, 42 mole equivalents of phosphatidylcholine and 18 mole equivalents of SM. The solutions were combined in a 1.5 ml glass vial, frozen (−70° C.), and lyophilized at −5 to −10° C. overnight. The resultant lyophilized cakes were rehydrated by addition of Dulbecco's phosphate buffered saline followed by gentle swirling of the contents. Formation of PALM was completed by incubating the PALM solution at 50° C. for 10 minutes. Some peptide complexes remained turbid upon heating and were also subjected to one cycle of freezing to −80° C. followed by thawing to room temperature in an attempt to obtain a clear solution. The qualities of the PALM preparations were evident in their appearance. A visually clear preparation indicated any nanoparticles that had formed were less than approximately 40 nm diameter, based on the Tyndall effect. Results are shown in Table 5.

TABLE 5
Stability of Peptide/Phospholipid Complexes by Visual Inspection

| | | Mole | | | | |
|---|---|---|---|---|---|---|
| | Mole | Ratio | Peptide | | | |
| Phospholipid | Ratio | C₁₆PTX/ | SEQ ID | SEQ ID | SEQ ID | SEQ ID |
| Content^{a} | PC/SM | PL | No. 25^{b} | No. 26 | No. 27 | No. 32 |
| POPC, SM | 70/30 | 0 | Clear | Clear | Clear | Clear |
| POPC, SM | 70/30 | 0.1 | Clear | Turbid | Turbid | Turbid |
| DOPC, SM | 70/30 | 0 | Clear | | Turbid | Turbid |
| DOPC, SM | 70/30 | 0.1 | Clear | | | Turbid |

^{a}1-palmitoyl-2-oleoyl phosphatidylcholine (POPC), 1,2-dioleoyl phosphatidylcholine (DOPC), egg sphingomyelin (SM), phosphatidylcholine (PC), phospholipids (PL), paclitaxel 2′-palmitate (C₁₆PTX)
^{b}Mole ratio of peptide to phospholipids was 1/4.

### Example 5. Exemplary PALM Preparation

Peptide having an amino acid sequence as set forth in SEQ ID NO:35, with acetate counterions, was custom synthesized by standard FMOC solid phase peptide synthesis. Stock solutions of the PALM components were prepared as follows. A 10 mM solution of peptide was prepared in tert-butyl alcohol (TBA)/water/acetic acid (80:20:7). Twenty millimolar solutions of POPC and SM were prepared in TBA/water (80/20). A 10 mM solution of XTT was in TBA/water (95:5).

PALM preparation was initiated by mixing 10 mM XTT (1 mol equivalents), plus 20 mM POPC (5.6 mol equivalents), plus 20 mM SM (2.4 mol eqivalents), plus 10 mM SEQ ID NO:35 (2 mol equivalent). The mixed volume was frozen by swirling in a dry ice/2-propanol bath and further frozen by placement in a −76° C. freezer for 1 hour. The mixture was lyophilized for 24 hr at −15° C. followed by an additional 20 hours at 15° C. A volume of sterile Dulbecco's phosphate-buffered saline was added to the lyophilizate to obtain 6.3 mM XTT (5.4 mg/ml PTX equivalents). The samples were briefly swirled to dissolve the cake and then placed in a 55° C. water bath for 30 minutes and finally briefly (˜10-20 sec) held in the sonic node of a water bath sonicator. The samples were filter-sterilized through 25 mm×0.2 μm PES filters and combined. The sterile preparation was distributed to pyrogen-free, glass vials that were sealed with butyl rubber stoppers with crimped aluminum tops and stored at 4° C.

### Example 6. SKOV-3 Growth Inhibition In Vitro

SKOV-3 cells were plated in 96-well tissue culture plates at 5,000 cells/well in 100 μl Dulbecco's minimally essential medium containing 10% fetal bovine serum and incubated in a 37° C. incubator containing 5% CO₂ in a humidified atmosphere. After 24 hr the medium was replaced with complete medium containing test articles. The highest concentration of paclitaxel in complete medium (10 μM) was prepared by addition of a 1 mM stock solution of paclitaxel in DMSO. Stock PALM preparations of 3 mM XTT in PALM were diluted into medium to a 50 μM concentration. Serial dilution in complete medium of these test solutions was conducted to obtain the test solutions at lower concentrations. Cells were incubated with test article solutions for 7 2hr. Next 20 μl of 5 mg/ml thiazolyl blue tetrazolium bromide in phosphate-buffered saline was added to all wells followed by a 4 hr incubation. All wells were washed with Dulbecco's phosphate buffered saline (with calcium/magnesium) and refilled with 100 μl DMSO. Plates were incubated at 37° C. for 30 minutes followed by determination of optical density in each well with a plate reader set to 590 nm. The concentration resulting in 50% growth inhibition (IC50) was determined by non-linear regression fit of the data to the logistic equation. The average optical density of the control wells was represented 100% growth.

### Example 7. Cytokine Production

SKOV-3 cells were plated in 96-well tissue culture plates at 10,000 cells/well in 100 μl Dulbecco's minimally essential medium containing 10% fetal bovine serum. After 24 hr the medium was removed. The cell layers were washed with Dulbecco's phosphate buffered saline (with calcium/magnesium). Wells were refilled with serum-free medium containing test articles. Test groups were: 1) no addition. 2) 10 μg/ml lipopolysaccharide. 3) 10 μM paclitaxel (added to medium from a stock solution in DMSO), and 4) 10 μM paclitaxel-equivalents of PALM (XTT) prepared with SEQ ID NO:25 peptide. Cells were incubated for 24 hr. Media were subsequently collected and centrifuged (1500 rpm microfuge). Supernatants were recovered and frozen for cytokine testing. Interleukin 6 (IL-6) content in the media was determined with the human IL-6 assay kit from RayBiotech Life (Peachtree Corners, GA).

### Example 8. SKOV-3 Xenografts in Mice

The protocol was approved by the Institutional Animal Care and Use Committee. Thirty, female, NU(NCr)-Foxn1nu nude mice (Harlan Laboratories) were housed in irradiated sterile IVC cages (up to 5 mice per cage) at 22-25° C. 40-60% humidity with 12 hours light and 12 hours darkness. Cages contained irradiated corncob bedding and sterile water. The diet was irradiation-sterilized, dry, granule food. Mice were acclimated for 7 days.

The SKOV-3 (ATCC) human ovarian tumor cells were maintained in vitro as a monolayer culture in McCoy's-5A medium supplemented with 10% fetal bovine serum at 37° C. in an atmosphere of 5% CO₂ in air. The cells were routinely sub-cultured twice weekly by trypsin-EDTA treatment (0.25% Trypsin-EDTA). Cells in an exponential growth phase were harvested and analyzed by GUAVA PCA flow cytometry for cell count and cell viability (99%) prior to xenotransplantation.

Thirty mice, aged 9-11 weeks, were inoculated subcutaneously at the flank region with SKOV-3 tumor cells (1.0×10⁶) in 0.1 ml of IxPBS mixed with Matrigel (1:1) for tumor development. Measurable tumors (50-100 mm3) developed after 7 days. Twenty-five animals with approximately 50-100 mm3 tumors (measured with electronic caliper) were selected for study and randomly placed into Groups 1-5 using randomized block design, as follows. First, the experimental animals were divided into 5 homogeneous blocks based on their tumor volume. Secondly, within each block, randomization of experimental animals to different groups was conducted.

Mice were checked daily for morbidity, mortality and any adverse effects of tumor growth and treatments on normal behavior such as mobility, visual estimation of food and water consumption, body weight gain/loss, eye/hair matting and any other abnormal effects.

Tail vein injections of test solutions were performed using a Genie Touch Syringe Pump (Kent Scientific). Terumo Surshield safety winged infusion sets (S25BLS, 25G×3/4) were used to access tail veins. A new syringe was used for each individual test article. All work was performed in a biological safety cabinet.

Dosing was performed on days 7, 11, 15, 19, 23, and 27. Day 0 was the day of xenotransplantation. All mice were dosed at 8 ml/kg. Dosing solutions groups were 17% cremophor EL/Ethanol (1:1) in saline (paclitaxel vehicle) (Group 1). 1.25 mg/ml paclitaxel in cremaphor EL/ethanol/saline (Group 2), 1 mg/ml paclitaxel equivalents of PALM (XTT) (Group 3), 2.5 mg/ml paclitaxel equivalents of PALM (XTT) (Group 4), and PALM without XTT at 1.25×the amount of PALM constituents of Group 4 (Group 5). PALM was prepared with SEQ ID NO:35 peptide.

Tumor volumes were measured on days 7, 12, 17, 22, 27, 32, 37 and 42 in two dimensions using an electronic caliper, and the volume will be expressed in mm³ using the formula: V=0.5 a×b² where a and b are the long and short diameters of the tumor, respectively.

### Example 9. Rat Chemotherapeutic Agent Induced Peripheral Neuropathy (CIPN)

Pharmaceutical grade Paclitaxel (PTX, Teva Pharmaceuticals) was diluted from a stock solution of 6 mg/ml in a 1:1 mixture of Cremaphore EL and ethanol to 1 mg/ml with saline. PTX was injected at 1 mg/kg Q2D×6. PALM, prepared with SEQ ID No:35 peptide and containing XTT, was administered at 1 mg/kg PTX equivalent dose and at 2.7 mg/kg PTX equivalent dose using the same dosing scheme as PTX. All drug and vehicle injections were performed i.p. on days 2. 4, 6, 8,10, and 12.

Male Sprague-Dawley rats in the control and experimental groups received equivalent volumes of solution. There were a total of 5 groups with a sample size of 10 animals per group. Group A received a cremophor EL/ethanol/saline solution equivalent to the dosing solution PTX was administered in, Group B received 1 mg/kg paclitaxel, Group C received phosphate-buffered saline (PALM vehicle), Group D received 1 mg/kg PTX equivalents XTT in PALM, Group E received 2.7 mg/kg PTX equivalents XTT in PALM.

Animals were housed in pairs, with each pair of animals belonging to the same group, to control for exposure of compound due to the excretion of paclitaxel (and possibly test compound) in urine and feces, in a temperature-controlled room on a 12:12 (7 am to 7 pm) light/dark cycle with free access to water and food. All procedures were approved by the Institutional Animal Care and Use Committee and adhered to the guidelines set forth by the Committee for Research and Ethical Issues of the International Association for the Study of Pain (Zimmerman, 1983).

To test for the onset of peripheral neuropathy, animals were examined for changes in mechanical paw withdrawal threshold (MPWT) values at baseline and then every other day for the duration of the 14-day protocol (Day 1 (Baseline), 3, 5, 7, 9, 11, 13). For this test, animals were placed within a Plexiglas chamber (20 cm×10.5 cm×40.5 cm) and allowed to habituate for 15 min. The chamber was positioned on top of a mesh screen so that mechanical stimuli could be administered to the plantar surface of both hind paws. Mechanical threshold measurements for each hind paw were obtained using an up/down motion with eight calibrated von Frey monofilaments (3.85, 5.68, 9.74, 18.39, 39.42, 77.30, 135.30, and 251.34 mN). Each trial began with a von Frey force of 9.74 mN delivered to the right hind paw for approximately 1 second, and then the left hind paw. If there was no withdrawal response, the next higher force was delivered. If a response was made, the next lower force was delivered. This procedure continued until no response was made at the highest force (251.34 mN) or until four stimuli were administered following the initial response. The withdrawal threshold for each paw was calculated using the following formula: [Xth]log=[vFr]log+ky, where [vFr] is the force of the last von Frey used. k=0.2593 which is the average interval (in log units) between the von Frey monofilaments. and y is a value that depends upon the pattern of withdrawal responses. If an animal did not respond to the highest von Frey hair, then y=1.00 and the mechanical paw withdrawal response for that paw is calculated to be 456.63 mN. The MPWT testing was performed across three trials per session and the withdrawal values were averaged over the three trials to determine the mean mechanical paw withdrawal threshold for each animal.

### Example 10. Preparation of PALM Containing the Fluorescent Dye DiI

A 40 μl aliquot of 10 mM peptide was combined with 56 μl of 20 mM POPC. 24 μl 20 mM SM(egg) and 16 μl 2.5 mM DiI in a small glass vial. The peptide and lipid solutions were prepared in 80% TBA/20% water. The DiI stock was prepared in 92% TBA/8% water. The combined solution was lyophilized and the resultant cake was rehydrated by addition of 0.2 ml of Dulbecco's phosphate buffered saline. The solution was briefly swirled, water bath sonicated (for approx. 15 sec.) and placed in a 50° C. heating block for 20 minutes.

### Example 11. Preparation of PALM Containing Miriplatin

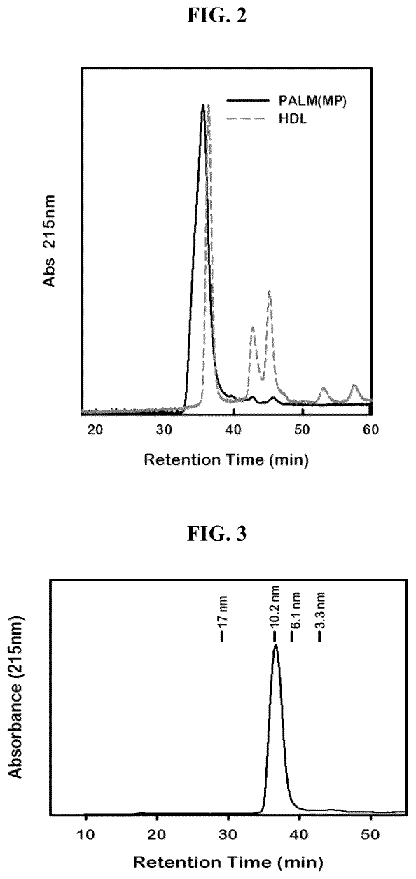

A 50 μL aliquot of 10 mM peptide having an amino acid sequence of SEQ ID NO:25 in 80% TBA/20% water corresponding to 2.5 mole equivalents of peptide was combined with 3 mole equivalents of POPC and 7 mole equivalents of egg SM from 40 mM and 20 mM stock solutions, respectively. made up of the same solvent mixture. To this was added 0).75 mole equivalents of miriplatin (MedKoo Biosciences, Raleigh. NC) from a 1 mM stock solution prepared with 100% TBA. The solution was lyophilized and the resultant cake was rehydrated by addition of 0.4 mL of 5% dextrose in water. The solution was briefly swirled, water bath sonicated (for approx. 15 sec.) and placed in a 50° C. heating block for 20 minutes. The resultant clear solution was passed through a 0.2 μm pore size, polyethersulfone, sterilization filter and stored at 4° C. Particle size analysis (Example 16) by DLS indicated a hydrodynamic mean diameter of 8 nm. SEC confirmed a single particle population comparable to HDL in size. The SEC chromatogram is shown in FIG. 2 (miriplatin (solid line), human HDL(dashed line)).

### Example 12. Preparation of PALM Containing Paclitaxel Cholesteryl Carbonate (XC)

A 50 μl aliquot of 10 mM of the peptide of SEQ ID NO:25 in 80% TBA/20% water corresponding to 2.5 mole equivalents of peptide was combined with 7 mole equivalents of POPC and 3 mole equivalents of egg SM from 20 mM stock solutions, made up of the same solvent mixture. To this was added I mole equivalent of XC from a 10 mM stock solution in 92% TBA/8% water. The solution was lyophilized and the resultant cake was rehydrated with Dulbecco's phosphate buffered saline to a final XC concentration of 1 mM. The hydrodynamic mean diameter of this preparation, determined by DLS, was 9 nm (Example 16). Size analysis by SEC indicated a single particle population principally 10 nm in diameter (FIG. 3).

### Example 13. Preparation of PALM Containing Paclitaxel δ-Tocotrienyl Carbonate (XTT)

A 50 μl aliquot of 10 mM of the peptide of SEQ ID NO:25 in 80% TBA/20% water corresponding to 2.5 mole equivalents of peptide was combined with 7 mole equivalents of POPC and 3 mole equivalents of egg SM from 20 mM stock solutions, made up of the same solvent mixture. To this was added I mole equivalent of XTT from a 10 mM stock solution in 92% TBA/8% water. The lyophilized cake was rehydrated with 0.4 ml of Dulbecco's phosphate buffered saline.

### Example 14. R4F is Unsuitable for Preparation of PALM Containing Paclitaxel δ-Tocotrienyl Carbonate (XTT)

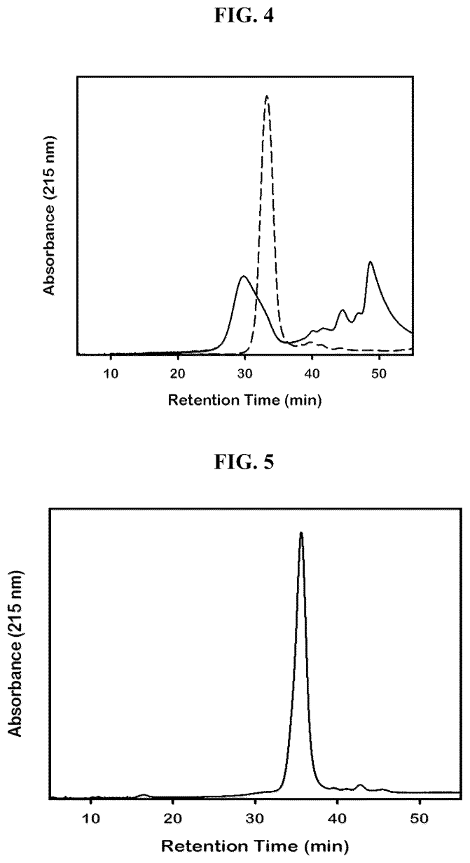

PALM preparation was conducted as in Example 13 with a peptide having an amino acid sequence of SEQ ID NO:25 and with the peptide R4F (Table 1). Unlike PALM made with the peptide of SEQ ID NO:25, which remained a clear solution at room temperature and 4° C., PALM containing the peptide R4F was a clear solution at room temperature but became a hazy gel at 4° C. The gel returned to clear liquid upon warming to room temperature. The PALM preparations were analyzed for size (Example 16). Dynamic light scattering indicated the PALM with the peptide of SEQ ID NO:25 had a mean hydrodynamic diameter of 8 nm (volume intensity). The same analysis for PALM with R4F showed 94% of particle population at a mean hydrodynamic diameter of 11 nm with the remainder at 32 nm. SEC confirmed the uniform size distribution of the PALM with the peptide of SEQ ID NO:25 (FIG. 4). In contrast, the PALM with peptide R4F showed a range of peaks eluting at sizes larger than the and less than that of SEQ ID NO:25 PALM. The lack of smaller particle detection by DLS is not surprising since sensitivity for particles below 7 nm is quite weak. These results indicate R4F is not a suitable peptide for PALM preparation.

### Example 15. Fenretinide is Loaded in PALM Prepared with the Peptide of SEQ ID NO:25

A 35 μl aliquot of 10 mM the peptide of SEQ ID NO:25 in 80% TBA/20% water corresponding to 2.5 mole equivalents of peptide was combined with 3 mole equivalents of POPC and 7 mole equivalents of egg SM from 40 mM and 20 mM stock solutions, respectively. made up in the same solvent mixture. Two mole equivalents of 20 mM fenretinide, in the same solvent mixture, were also added. The solution was lyophilized and the resultant cake was rehydrated with 0.325 ml phosphate buffered saline. The solution became clear within 20 min at 50° C. Analysis by SEC (Example 16) indicated all components eluted as a single peak in the 8 nm-10 nm diameter range (FIG. 5).

### Example 16. Determination of PALM Size

The size and size uniformity of the PALM preparations was determined by DLS and SEC. Sizes based on hydrodynamic mean diameters were determined by DLS with a Nicomp 370 particle size analyzer. The analyzer was calibrated with latex standards. Particle sizes referred to herein and in the claims are calculated by DLS as described above unless clearly indicated otherwise.

The relative hydrodynamic size of PALM particles was also determined by SEC with a GE Superose 6 Increase column. (10×300 mm) connected to a Beckman/Coulter Model 126 pump and a Model 128 diode array detector. The mobile phase (150 mM NaCl, 6 mM NaPO₄ (pH 7.4)) flow rate was 0.5 mL/min. The eluent was monitored at 215 and 280 nm wavelengths. System performance was confirmed by injection of protein molecular weight standards (FIG. 3).

### Example 17. SR-BI Selectivity of PALM in BHK(SR-BI) Cells

SR-BI interaction studies are done with BHK(SR-BI) cells stably transfected with an inducible human SR-BI gene by means of the GeneSwitch™ System (Invitrogen) (Vickers et al. (2011) Nat. Cell Biol. 13:423-433) . The cells were plated (96-well plate) (8000 cells/well) in growth medium (Dulbecco's modified Eagle medium containing 10% fetal bovine serum) containing 200 μg/ml each of zeocin and hygromycin. After 24 hours incubation, the growth medium was removed and replaced with 0.2% bovine serum albumin in Dulbecco's modified Eagle medium. The medium of cells to be induced for SR-BI expression also contained 10 nM mifepristone, added from a DMSO stock solution. DMSO alone was added to the medium of uninduced cells. The induction medium was removed after 24 hours and replaced with medium containing DiI-labeled PALM (32 μg peptide/ml) or DiI-labeled HDL (19 μg protein/mL) (Kalen Biomedical, Montgomery Village, MD). The test media were prepared by diluting an aliquot of DiI-labeled PALM (Example 10) or the DiI-labeled HDL in 0.2% bovine serum albumin in Dulbecco's modified Eagle medium. The solutions were passed through 0.2 μm pore size, polyethersulfone, sterilization filters before use. The cells were incubated for 4 hours. Next, the cells were washed 3 times with 0.1% albumin in Dulbecco's phosphate buffered saline (with calcium and magnesium). The last wash was replaced with 200 μl/well of t-butanol/water (95%/5%). The covered plate was left to stand at room temp (20-21° C.) for 30 min with occasional shaking. The fluorescence in each well was detected at 520 nm excitation and 580 nm emission with a 550 nm cutoff filter on a Molecular Dynamics Gemini fluorescence plate reader (FIG. 9).

TABLE 6
DiI Uptake from HDL and from PALM Prepared with Various
Peptides by BHK(SR-BI) Cells Depends on SR-BI Expression

| | Un-induced | Induced | |
|---|---|---|---|
| | DiI Uptake ^{a} | DiI Uptake | Increase Over |
| | (pmol/ug/ml) | (pmol/ug/ml) | Un-induced |
| HDL ^{b} | 0.13 ± 0.02 | 0.86 ± 0.03 | 561% |
| SEQ ID NO: 3 | 0.16 ± 0.01 | 0.29 ± 0.03 | 88% |
| SEQ ID NO: 5 | 0.54 ± 0.03 | 0.93 ± 0.03 | 73% |
| SEQ ID NO: 25 | 0.24 ± 0.01 | 0.83 ± 0.02 | 245% |
| SEQ ID NO: 26 | 0.49 ± 0.04 | 0.81 ± 0.04 | 65% |
| SEQ ID NO: 27 | 0.34 ± 0.01 | 0.66 ± 0.02 | 90% |
| SEQ ID NO: 32 | 0.28 ± 0.02 | 0.90 ± 0.03 | 221% |
| SEQ ID NO: 35 | 0.35 ± 0.01 | 1.26 ± 0.06 | 264% |

^{a} Amount of DiI taken up by cells relative to protein (HDL) or peptide (PALM) concentration. The average (n = 4) and standard error of the mean are shown.
^{b} HDL DiI content was 21 pmol/ug protein. PALM DiI content was 40 pmol/ug peptide. HDL concentration was 19 μg/ml. PALM peptide concentrations were 32 μg/ml.

### Example 18. Quantification of Paclitaxel

Paclitaxel, XTT and XC are extracted from aqueous samples by mixing 1 volume aqueous sample with 4 volumes of ethyl acetate/acetone/methanol (70/30/5 v/v). The upper organic layer, obtained after shaking and centrifugation, is collected, dried by solvent evaporation and vacuum, and re-dissolved in HPLC mobile phase (methanol/water (65/35 v/v)). A 20 μL aliquot of reconstituted sample is injected on an HPLC at a flow rate of 1.2 ml/minute through a Macherey-Nagel column (4×250 mm with Nucleosil 10-5 C18) and detected with a UV detector at 230 nm wavelength.

### Example 19. PALM Containing Miriplatin Inhibits PC-3 Cell Growth as Well as Cisplatin

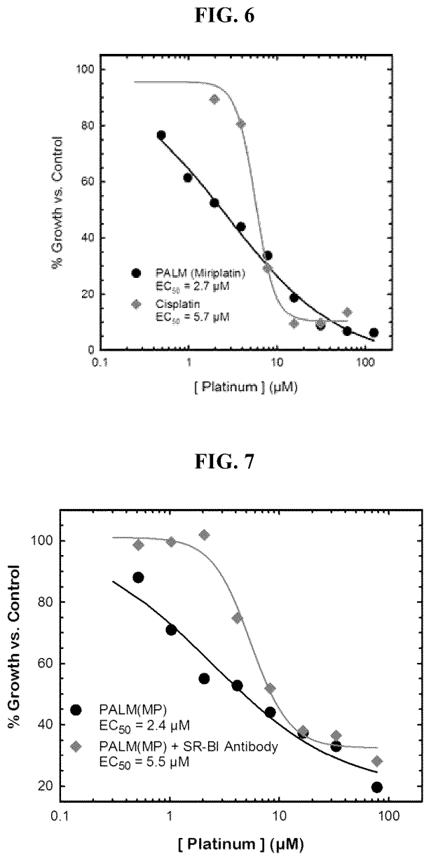

PC-3cells (American Type Culture Collection, CRL-1435) were seeded in 96-well plates at a density of 5×10³ cells per well (100 μL) and grown till approximately 70% confluence (24 hour) in growth medium composed of F-12K medium supplemented with 10% fetal bovine serum. Next, growth medium was replaced by either 100 μL fresh growth medium (control) or by growth medium supplemented with various concentrations of cisplatin (e.g. 0 μM and 0.1 to 100 μM final concentration in medium) added from 100-fold concentrated stock solutions prepared in 5% dextrose or with equivalent amounts of miriplatin in PALM, as prepared in Example 11. Each condition was tested in triplicate. Plates were incubated for 48 hours. Cell viability was assayed with the thiazolyl blue tetrazolium bromide (MTT) assay by adding 20 μl of 5 mg/ml MTT in Dulbecco's phosphate buffered saline (with calcium and magnesium) and incubating for 3 hours. Next, media were carefully removed and replaced by 200 μL of dimethylsulfoxide (DMSO). The plates were agitated gently for 15 minutes on an orbital shaker. The absorbance of each well was read at 570 nm. The concentration resulting in 50% growth inhibition (IC₅₀) was determined by non-linear regression fit of the data to the logistic equation. The average absorbance of the control wells represented 100% growth (FIG. 6).

### Example 20. SR-BI Antibody Attenuates PC-3 Cell Growth Inhibition by PALM Containing Miriplatin

PC-3 cells were grown as in Example 19. Cells to be tested in the presence of SR-BI antibody (Novus Biologics, NB400-113) were preincubated for 1 hr in growth medium containing a 1/400 dilution of stock antibody solution. Next, all media were removed and replaced with growth medium containing the indicated amounts of platinum compounds, prepared as in Example 13. The growth media with PALM(MP) for the antibody-treated cells contained antibody at a 1/400 dilution of stock antibody solution. The cells were incubated for 5 hr. Next, all media were removed; the cells were washed one time with medium and then incubated for a further 43 hours in growth medium. Cell survival was determined by MTT assay as in Example 19 (FIG. 7).

### Example 21. XTT in PALM is More Active Than XC in PALM in Blocking SKOV-3 Cell Growth XC

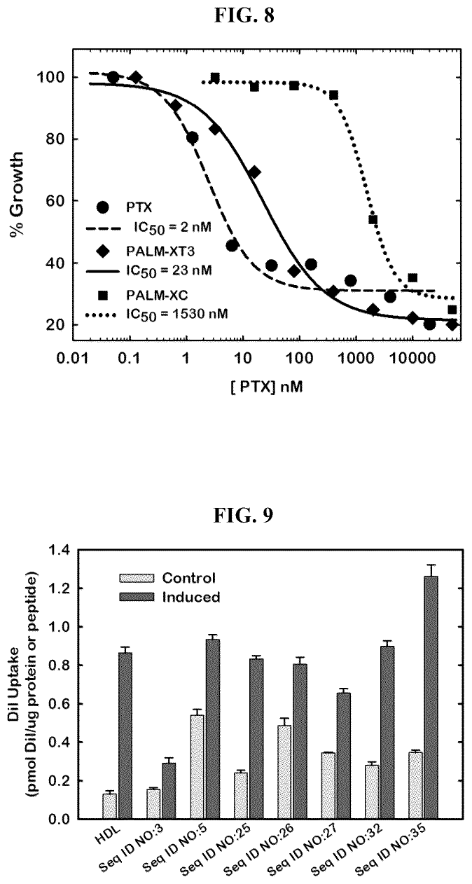

SKOV-3 ovarian cancer cells (American Type Culture Collection, HTB-77) were seeded in 96-well plates at a density of 5×10³ cells per well (100 μL) and grown till approximately 70% confluence (24 hour) in growth medium composed of McCoy's medium supplemented with 10% fetal bovine serum. Next, growth medium was replaced by either 100 μL fresh growth medium (control) or by growth medium supplemented with various concentrations of paclitaxel. PALM(XC) or PALM(XTT). A test solution of 20 μM paclitaxel was prepared by dilution of a 5 mM stock solution of paclitaxel in DMSO into growth medium followed by filter sterilization (0.2 μm filter). An aliquot of the 20 μM solution was diluted 5-fold in growth medium to obtain 4 μM paclitaxel. The 5-fold dilution process was continued with the 4 μM to obtain an 800 nM paclitaxel solution. This process was continued until a concentration of 0.051 nM paclitaxel in growth medium had been obtained. Four 100 μL aliquots of each of the 9 solutions thus obtained were applied to separate wells containing cells. A similar but modified process was used for preparation of PALM(XC) and PALM(XTT) test solutions. The highest concentration tested was 50 μM, which was prepared by dilution of 1 mM preparations of PALM(XC) and PALM(XTT) in growth medium followed by filter sterilization. The lowest concentration obtained in the process of 5-fold dilution of each newest dilution repeated 8 times was 0.13 nM. Cells were incubated with the test solutions for 72 hours. At the end of this period cell viability was determined by MTT assay, as in Example 19. (FIG. 8).

### Example 22. Inhibition of BHK(SR-BI) Cell Growth by PALM(XTT) is SR-BI-Dependent

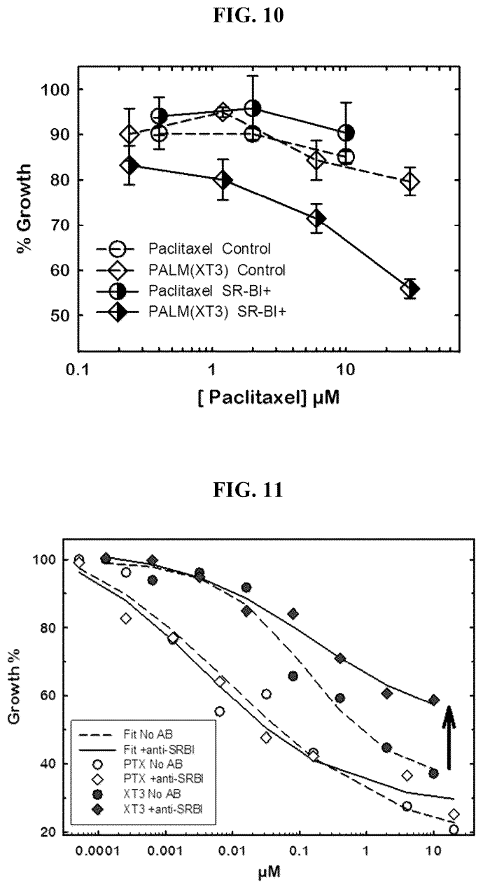

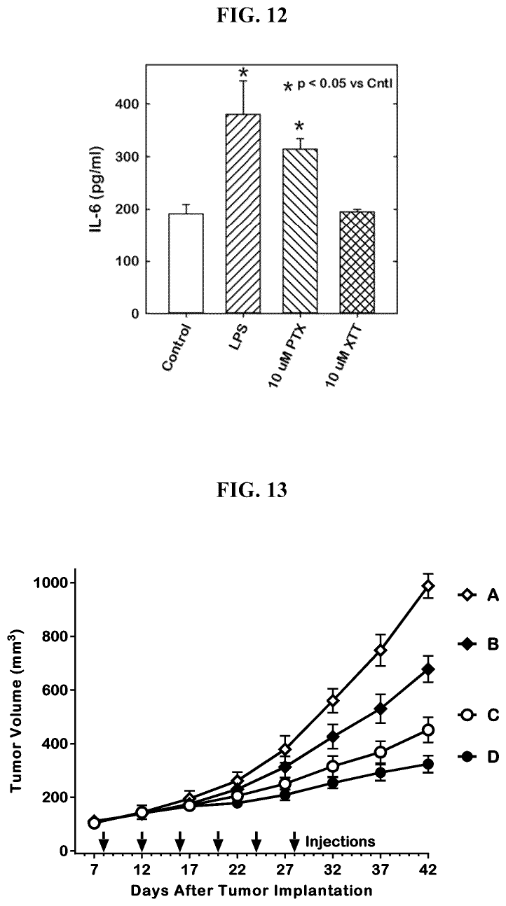

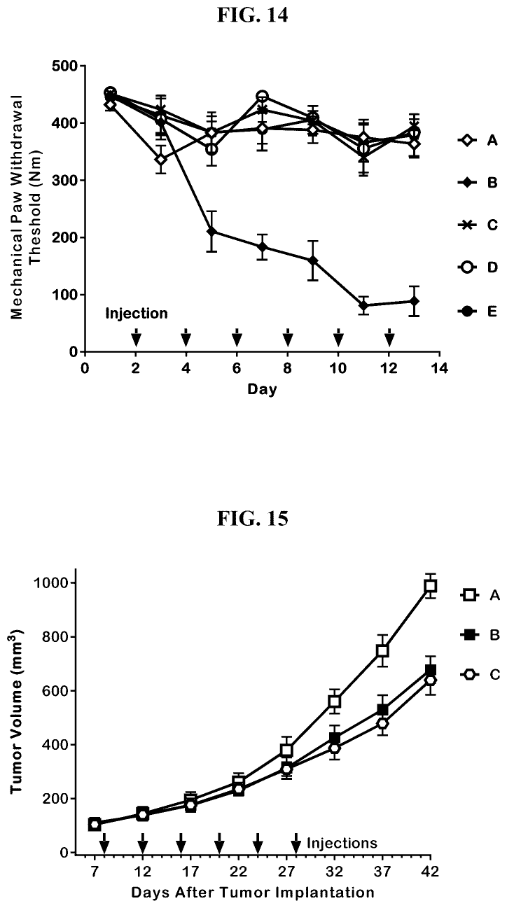

BHK(SR-BI) cells were plated (3000 cells/well) in 96-well plates with growth medium (Dulbecco's modified Eagle medium containing 10% fetal bovine serum and 200 μg/ml each of zeocin and hygromycin) and incubated 24 hours. Growth medium was replaced with 0.2% bovine serum albumin in Dulbecco's modified Eagle medium containing either 10 nM mifepristone (induced), added from a DMSO stock solution, or the equivalent amount of only DMSO (Control). Cells were incubated for 24 hours. Next, media were replaced with PTX or PALM(XTT) in 0.2% bovine serum albumin in Dulbecco's modified Eagle medium, at the indicated concentrations, and the cells were incubated for 12 hours. Those media were then replaced by normal growth medium and the cells were incubated for 36 hr more. Percent cell growth relative to cells without test agent was determined by MTT assay (FIG. 10).

### Example 23. δ-Tocotrienyl (N⁴)-Gemcitabine Carbamate

The hydroxyl groups in gemcitabine are protected by conversion to tert-butoxycarbonyl (BOC) esters with di-tert-butyl dicarbonate following the procedure of Guo and Gallo (J. Org. Chem. 1999, 64, 8319) to yield (1)

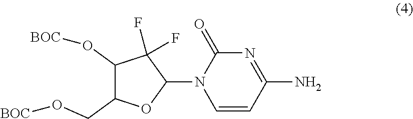 [SMILES: CC(C)(C)OC(=O)OCC1OC(n2ccc(N)nc2=O)C(F)(F)C1OC(=O)OC(C)(C)C]

Compound 4 is dissolved in anhydrous dichloromethane to a final concentration of 0.2M compound (4). For every mole of compound (4) in solution, 1.2 mole equivalents of compound (2) at 0.5M concentration in methylene chloride and 3 mole equivalents of DMAP are combined at rt. The mixture is stirred at room temperature for 24 h. The resultant product is deprotected with trifluoracetic acid, as referenced. Pure compound is obtained by flash column chromatography using dichloromethane and methanol eluent, beginning with 100% dichloromethane and gradually increasing the concentration to 10% methanol to yield the titled compound (5).

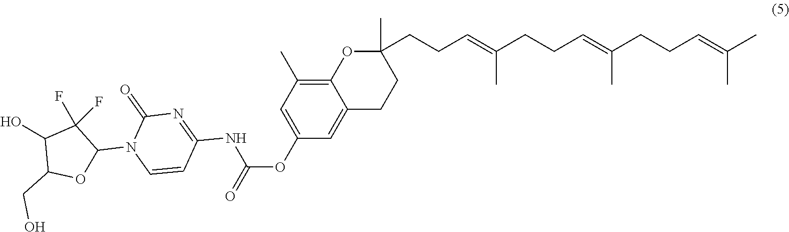 [SMILES: CC(C)=CCC/C(C)=C/CC/C(C)=C/CCC1(C)CCc2cc(OC(=O)Nc3ccn(C4OC(CO)C(O)C4(F)F)c(=O)n3)cc(C)c2O1]

### Example 24. Synthesis of (N⁴)-Gemcitabine Carbamates with the α,β or γ-Tocotrienol Isomers is Performed Similarly to Example 24

### Example 25. Cholesteryl (N⁴)-Gemcitabine Carbamate

The synthesis of cholesteryl (N⁴)-gemcitabine carbamate (6) is performed in the same manner as described in Example 25 with the exception that compound (4) is reacted with cholesterol chloroformate (commercially available) and deprotected as in Example 19 to yield the titled compound (6).

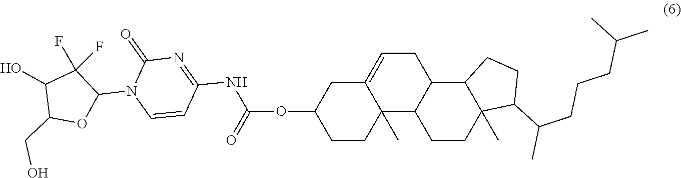 [SMILES: CC(C)CCCC(C)C1CCC2C3CC=C4CC(OC(=O)Nc5ccn(C6OC(CO)C(O)C6(F)F)c(=O)n5)CCC4(C)C3CCC12C]

### Example 26. Paclitaxel Linked to Fatty Alcohols Via Succinic and Diglycolic Acids

Synthesis of paclitaxel linked to fatty alcohol via a succinate or diglycolate di-ester link is accomplished by reacting fatty alcohol with 4-(dimethylamino)pyridine and succinic anhydride or diglycolic anhydride in anhydrous pyridine with constant stirring for 24 h at room temperature. The reaction is quenched with 0.1 N HCl in dichloromethane. The product is obtained by preparative TLC or flash column chromatography with ethyl acetate in petroleum ether. The alcohol-succinic acid or -diglycolic acid conjugate is combined with 4-(dimethylamino)pyridine and N-(3-dimethylaminopropyl)-N′-ethylcarbodiimide in dry dichloromethane. Paclitaxel is added into the reaction mixture. After 24 h, the reaction is quenched with water and extracted with dichloromethane. The product is obtained by preparative TLC using ethyl acetate/heptanes (50:50) as eluent.

### Example 27. Effect of SR-BI Antibody on PALM(XTT) Cytotoxicity in SKOV-3 Cells

SKOV-3 were plated and incubated for 24 hour, as in Example 16. Next, growth medium was replaced with serum-free medium containing 0.5% albumin and the indicated concentrations of test agents, with or without anti-SRBI ( 1/250 dilution) (NB400-113, Novus Biologicals). The cells were incubated 12 hr. Next, the cells were washed with serum-free medium containing 0.5% albumin and grown a further 60 hour in growth medium. Cell growth was detected by MTT assay (FIG. 11).

While a number of embodiments of this disclosure are described, it is apparent that the basic examples may be altered to provide other embodiments that use or encompass methods and processes of this invention. The embodiments and examples are for illustrative purposes and are not to be interpreted as limiting the disclosure, but rather, the appended claims define the scope of this invention.

What is claimed is:

1-34. (canceled)

35. A method for the treatment or prevention of Chemotherapy-Induced Peripheral Neuropathy (CIPN) in a cancer patient treated with, or to be treated with, a chemotherapeutic agent causing CIPN, the method comprising:
administering a therapeutically effective amount of a composition containing a peptide amphiphile lipid micelle (PALM) nanoparticle to the cancer patient, the PALM nanoparticle comprising a PALM containing the chemotherapeutic agent causing CIPN, and wherein the PALM comprises:
a) i) a peptide, wherein the peptide in the PALM comprises the amino acid sequences: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀ wherein: X₁ is the amino acid D; X₂ and X₂₀ are each the amino acid V or Aib; X₃, X₆, X₁₀ and X₁₃ are each an amino acid independently selected from the group consisting of L and F; X₄, X₁₂ and X₁₉ are each the amino acid Q; X₅ is an amino A or Aib; X₇, X₁₆ and X₁₈ are each the amino acid K; X₈ and X₁₅ are each the amino acid E; X₉ and X₁₄ are each an amino acid independently selected from the group consisting of A, L, F and Aib; X₁₁ is an amino acid selected from the group consisting of A, Aib and N; and X₁₇ is an amino acid selected from the group consisting of W, F and L; or
a) ii) a peptide, wherein the peptide in the PALM comprises the amino acid sequences: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀, wherein: X₁ is an amino acid selected from the group consisting of D and E; X₂ and X₂₀ are each an amino acid independently selected from the group consisting of V, Aib, I, and L; X₃, X₆, X₁₀ and X₁₃ are each an amino acid independently selected from the group consisting of L, I, V, W, Y, Aib, Amv and F; X₄, X₁₂ and X₁₉ are each an amino acid independently selected from the group consisting of Q and N; X₅, X₁₆ and X₁₈ are each an amino acid independently selected from the group consisting of K, R, H and Orn; X₇ is selected from the group consisting of A, G, S, V, Aib, and Amv; X₈ and X₁₅ are independently selected from the group consisting of the amino acid E and D; X₉ and X₁₄ are an amino acid independently selected from the group consisting of A, G, S L, F, V, Amv, and Aib; X₁₁ is an amino acid selected from the group consisting of A, G, S, Aib, Amv, V and N; and X₁₇ is an amino acid selected from the group consisting of W, F, Y, I, V, and L, (SEQ ID NO:24), wherein the peptide is optionally acylated at the N-terminus, amidated at the C-terminus, or both acylated at the N-terminus and amidated at the C-terminus and the peptide is from 20 to 24 amino acid in length;
b) a lipid component comprising sphingomyelin and one or more additional phospholipids; and
c) a cargo molecule that is a compound conjugate having the formula (I):
A—R—L—X (formula I)
wherein A is an agent having a hydroxyl or an amine group; R is the hydroxyl or the amine group of the agent; L is a linker; and X is an anchor moiety.

36. The method of claim 35, wherein the peptide in the PALM consists of an amino acid sequence selected from the group consisting of SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; or SEQ ID NO:23, wherein the peptide is optionally acylated at the N-terminus, amidated at the C-terminus, or both acylated at the N-terminus and amidated at the C-terminus and the peptide is 20 amino acids in length.

37. The method of claim 35, wherein the peptide in the PALM consists of an amino acid sequence selected from the group consisting of SEQ ID NO:25; SEQ ID NO:26; SEQ ID NO:27; SEQ ID NO:28; SEQ ID NO:29; SEQ ID NO:30; SEQ ID NO:31, SEQ ID NO:32; SEQ ID NO:33; SEQ ID NO:34; SEQ ID NO:35, and SEQ ID NO:36, wherein the peptide is optionally acylated at the N-terminus, amidated at the C-terminus, or both acylated at the N-terminus and amidated at the C-terminus and the peptide is 20 amino acids in length.

38. The method of claim 35, wherein the one or more additional phospholipids is selected from the group consisting of phosphatidylcholine, polyethylene glycol-phosphatidylethanolamine (PEG-PE), phosphatidylethanolamine, phosphatidylglycerol, phosphatidylserine, phosphatidylinositol, cardiolipin, or any combination thereof.

39. The method of claim 38, wherein the one or more additional phospholipid comprises a phosphatidylcholine, wherein the phosphatidylcholine is 1-palmitoyl-2-oleoyl-phosphatidylcholine (POPC).

40. The method of claim 35, wherein the PALM nanoparticle is administered before the onset of CIPN, or during the CIPN, or after the amelioration of CIPN, or any combination thereof.

41. The method of claim 35, wherein the composition comprises at least one cargo molecule that is a compound conjugate having the formula (I):
A—R—L—X (formula I)
wherein A is an agent having a hydroxyl or an amine group; R is the hydroxyl or the amine group of the agent; L is a linker; and X is an anchor moiety,
wherein X is an anchor moiety selected from the group consisting of cholesterol, α-tocotrienol, β-tocotrienol, γ-tocotrienol, δ-tocotrienol, δ-tocopherol, cholecalciferol, or ergocalciferol;
wherein R is a hydroxy group and the anchor moiety is covalently bonded to agent by a carbonate ester bond or R is an amine group and the anchor moiety is covalently bonded to the agent by a carbamate ester bond.

42. The method of claim 41, wherein X the anchor moiety is cholesterol, or the anchor moiety is δ-tocotrienol, or the anchor moiety is δ-tocopherol.

43. The method of claim 41, further comprising administering one or more additional chemotherapeutic agent or agents, wherein the one or more additional chemotherapeutic agent or agents are compatible with the chemotherapeutic agent causing CIPN is contained by the PALM.

44. The method of claim 41, wherein the chemotherapeutic agent contained by the PALM is a chemotherapeutic agent which causes, is likely to cause, or is associated with the CIPN of the cancer patient.

45. The method of claim 41, wherein the agent is a chemotherapeutic agent causing CIPN or a chemotherapeutic agent is selected from the group consisting of bortezomib, carboplatin, cisplatin, miriplatin, gemcitabine, misonidazole, oxaliplatin, procarbazine, thalidomide, docetaxel, hexamethylmelamine, paclitaxel, vincristine, vinblastine, vinorelbine, ixabepilone, eribulin, mertansine.

46. The method of claim 45, wherein the agent is a chemotherapeutic agent causing CIPN is selected from the group consisting of paclitaxel, vinblastine or vincristine.

47. The method of claim 46, wherein the agent is a chemotherapeutic agent causing CIPN is the 2′-cholesteryl carbonate ester of paclitaxel, the delta-tocotrienyl carbamate ester of paclitaxel, or the cholesteryl carbonate ester of vincristine.

48. The method of claim 35, wherein the cancer patient has a cancer that is selected from the group consisting of ovarian cancer, cervical cancer, endometrial cancer, colorectal cancer, prostate cancer, breast cancer, pancreatic cancer, head & neck cancer, testicular cancer, leukemia, neuroblastoma, Hodgkin's lymphoma, non-Hodgkin's lymphoma, and non-small cell lung cancer.

49. The method of claim 48, wherein the cancer is selected from the group consisting of ovarian cancer, breast cancer, and non-small cell lung cancer.

50. The method of claim 37, wherein the peptide in the PALM is selected from the group consisting of SEQ ID NO:25; SEQ ID NO:32; SEQ ID NO:35 and SEQ ID NO:36.

51. The method of claim 50, wherein the agent is a chemotherapeutic agent causing CIPN or a chemotherapeutic agent is selected from the group consisting of bortezomib, carboplatin, cisplatin, miriplatin, gemcitabine, misonidazole, oxaliplatin, procarbazine, thalidomide, docetaxel, hexamethylmelamine, paclitaxel, vincristine, vinblastine, vinorelbine, ixabepilone, eribulin, mertansine.

52. The method of claim 51, wherein the agent is a chemotherapeutic agent causing CIPN is selected from the group consisting of paclitaxel, vinblastine or vincristine.

53. The method of claim 52, wherein the agent is a chemotherapeutic agent causing CIPN is the 2′-cholesteryl carbonate ester of paclitaxel, the delta-tocotrienyl carbamate ester of paclitaxel, or the cholesteryl carbonate ester of vincristine.

54. The method of claim 52, wherein the cancer patient has a cancer that is selected from the group consisting of ovarian cancer, cervical cancer, endometrial cancer, colorectal cancer, prostate cancer, breast cancer, pancreatic cancer, head & neck cancer, testicular cancer, leukemia, neuroblastoma, Hodgkin's lymphoma, non-Hodgkin's lymphoma, and non-small cell lung cancer.


SEQUENCE LISTING (72 sequences)

SEQ ID NO: 1 - protein, 20 aa, Artificial Sequence
  synthetic peptide
  MISC_FEATURE (2)..(2): V or Aib
  MISC_FEATURE (3)..(3): L or F
  MISC_FEATURE (5)..(5): A or Aib
  MISC_FEATURE (6)..(6): L or F
  MISC_FEATURE (9)..(9): A, L, F or Aib
  MISC_FEATURE (10)..(10): L or F
  MISC_FEATURE (11)..(11): A, Aib or N
  MISC_FEATURE (13)..(13): L or F
  MISC_FEATURE (14)..(14): A, L, F or Aib
  MISC_FEATURE (17)..(17): W, F or L
  MISC_FEATURE (20)..(20): V or Aib
DXXQXXKEXXXQXXEKXKQX

SEQ ID NO: 2 - protein, 20 aa, Artificial Sequence
  Synthetic peptide
DVFQALKELFAQLLEKWKQV

SEQ ID NO: 3 - protein, 20 aa, Artificial Sequence
  synthetic peptide
  MISC_FEATURE (5)..(5): Aib
DVFQXLKELFNQLLEKWKQV

SEQ ID NO: 4 - protein, 20 aa, Artificial Sequence
  synthetic peptide
  MISC_FEATURE (5)..(5): Aib
DVFQXLKELLAQLLEKFKQV

SEQ ID NO: 5 - protein, 20 aa, Artificial Sequence
  synthetic peptide
  MISC_FEATURE (5)..(5): Aib
DVFQXLKELLNQLLEKFKQV

SEQ ID NO: 6 - protein, 20 aa, Artificial Sequence
  synthetic peptide
  MISC_FEATURE (5)..(5): Aib
  MISC_FEATURE (14)..(14): Aib
DVFQXLKELLNQLXEKFKQV

SEQ ID NO: 7 - protein, 20 aa, Artificial Sequence
  synthetic peptide
  MISC_FEATURE (5)..(5): Aib
  MISC_FEATURE (14)..(14): Aib
DVFQXLKELLNQLXEKWKQV

SEQ ID NO: 8 - protein, 20 aa, Artificial Sequence
  synthetic peptide
DVFQALKELLAQLLEKFKQV

SEQ ID NO: 9 - protein, 20 aa, Artificial Sequence
  synthetic peptide
DVFQALKELLNQLLEKFKQV

SEQ ID NO: 10 - protein, 20 aa, Artificial Sequence
  synthetic peptide
  MISC_FEATURE (5)..(5): Aib
DVFQXLKELFAQLLEKWKQV

SEQ ID NO: 11 - protein, 20 aa, Artificial Sequence
  synthetic peptide
  MISC_FEATURE (5)..(5): Aib
DVFQXLKELFNQLLEKWKQV

SEQ ID NO: 12 - protein, 20 aa, Artificial Sequence
  synthetic peptide
  MISC_FEATURE (5)..(5): Aib
DVFQXLKELFNQLLEKFKQV

SEQ ID NO: 13 - protein, 20 aa, Artificial Sequence
  synthetic peptide
  MISC_FEATURE (14)..(14): Aib
DVFQALKELFAQLXEKWKQV

SEQ ID NO: 14 - protein, 20 aa, Artificial Sequence
  synthetic peptide
  MISC_FEATURE (14)..(14): Aib
DVFQALKELFNQLXEKWKQV

SEQ ID NO: 15 - protein, 20 aa, Artificial Sequence
  synthetic peptide
  MISC_FEATURE (14)..(14): Aib
DVFQALKELFNQLXEKFKQV

SEQ ID NO: 16 - protein, 20 aa, Artificial Sequence
  synthetic peptide
DVFQAFKEAFAQLFEKWKQV

SEQ ID NO: 17 - protein, 20 aa, Artificial Sequence
  synthetic peptide
  MISC_FEATURE (9)..(9): Aib
DVFQAFKEXFAQLFEKWKQV

SEQ ID NO: 18 - protein, 20 aa, Artificial Sequence
  synthetic peptide
  misc_feature (5)..(5): Aib
  MISC_FEATURE (9)..(9): Aib
DVFQXFKEXFAQLFEKWKQV

SEQ ID NO: 19 - protein, 20 aa, Artificial Sequence
  synthetic peptide
  MISC_FEATURE (11)..(11): Aib
DVFQAFKEAFXQLFEKWKQV

SEQ ID NO: 20 - protein, 20 aa, Artificial Sequence
  synthetic peptide
  MISC_FEATURE (9)..(9): Aib
  MISC_FEATURE (11)..(11): Aib
DVFQAFKEXFXQLFEKWKQV

SEQ ID NO: 21 - protein, 20 aa, Artificial Sequence
  synthetic peptide
  MISC_FEATURE (5)..(5): Aib
  MISC_FEATURE (9)..(9): Aib
  MISC_FEATURE (11)..(11): Aib
DVFQXFKEXFXQLFEKWKQV

SEQ ID NO: 22 - protein, 20 aa, Artificial Sequence
  synthetic peptide
DVFQALKELFNQLLEKWKQV

SEQ ID NO: 23 - protein, 20 aa, Artificial Sequence
  synthetic peptide
  MISC_FEATURE (5)..(5): Aib
DVFQXLKELLNQLLEKLKQV

SEQ ID NO: 24 - protein, 20 aa, Artificial Sequence
  Synthetic peptide
  MISC_FEATURE (1)..(1): D or E
  MISC_FEATURE (2)..(2): V, Aib, I, L
  MISC_FEATURE (3)..(3): L, I, V, W, Y, Aib, Amv, or F
  MISC_FEATURE (4)..(4): Q or N
  MISC_FEATURE (5)..(5): K, R, H, or Orn
  MISC_FEATURE (6)..(6): L, I, V, W, Y, Aib, Amv, F
  MISC_FEATURE (7)..(7): A, G, S, V, Aib, or Amv
  MISC_FEATURE (8)..(8): D or E
  MISC_FEATURE (9)..(9): A, G, S L, F, V, Amv, or Aib
  MISC_FEATURE (10)..(10): L, I, V, W, Y, Aib, Amv, F
  MISC_FEATURE (11)..(11): Q or N
  MISC_FEATURE (12)..(12): Q or N
  MISC_FEATURE (13)..(13): L, I, V, W, Y, Aib, Amv, or F
  MISC_FEATURE (14)..(14): A, G, S, L, F, V, Amv, or Aib
  MISC_FEATURE (14)..(14): A, G, S, L, F, V, Amv, or Aib
  MISC_FEATURE (15)..(15): E or D
  MISC_FEATURE (16)..(16): K, R, H, or Orn
  MISC_FEATURE (17)..(17): L, I, V, W, Y, or F
  MISC_FEATURE (18)..(18): K, R, H, or Orn
  MISC_FEATURE (19)..(19): Q or N
  MISC_FEATURE (20)..(20): V,  Aib, I, L
XXXXXXXXXXXXXXXXXXXX

SEQ ID NO: 25 - protein, 20 aa, Artificial Sequence
  synthetic peptide
  MISC_FEATURE (7)..(7): Aib
DVFQKLXELFNQLLEKWKQV

SEQ ID NO: 26 - protein, 20 aa, Artificial Sequence
  synthetic peptide
DVFQKLVELFNQLLEKWKQV

SEQ ID NO: 27 - protein, 20 aa, Artificial Sequence
  synthetic peptide
  MISC_FEATURE (3)..(3): Aib
DVXQKLFELFNQLLEKWKQV

SEQ ID NO: 28 - protein, 20 aa, Artificial Sequence
  synthetic peptide
  MISC_FEATURE (7)..(7): Aib
DVFQKLXELFNQLLEKFKQV

SEQ ID NO: 29 - protein, 20 aa, Artificial Sequence
  synthetic peptide
DVFQKLVELFNQLLEKFKQV

SEQ ID NO: 30 - protein, 20 aa, Artificial Sequence
  synthetic peptide
  MISC_FEATURE (3)..(3): Aib
DVXQKLFELFNQLLEKFKQV

SEQ ID NO: 31 - protein, 20 aa, Artificial Sequence
  synthetic peptide
  misc_feature (7)..(7): Aib
DVLQKFXELFNQLLEKWKQV

SEQ ID NO: 32 - protein, 20 aa, Artificial Sequence
  synthetic peptide
  MISC_FEATURE (3)..(3): Aib
DVXQKFLELFNQLLEKWKQV

SEQ ID NO: 33 - protein, 20 aa, Artificial Sequence
  synthetic peptide
  MISC_FEATURE (9)..(9): Aib
DVFQKLLEXFNQLLEKWKQV

SEQ ID NO: 34 - protein, 20 aa, Artificial Sequence
  synthetic peptide
  MISC_FEATURE (7)..(7): Aib
  MISC_FEATURE (13)..(13): Aib
DVFQKLXELFNQXLEKWKQV

SEQ ID NO: 35 - protein, 20 aa, Artificial Sequence
  synthetic peptide
  MISC_FEATURE (7)..(7): Aib
  MISC_FEATURE (14)..(14): Aib
DVFQKLXELFNQLXEKWKQV

SEQ ID NO: 36 - protein, 20 aa, Artificial Sequence
  synthetic peptide
  MISC_FEATURE (2)..(2): Aib
  MISC_FEATURE (7)..(7): Aib
  MISC_FEATURE (14)..(14): Aib
DXFQKLXELFNQLXEKWKQV

SEQ ID NO: 37 - protein, 20 aa, Artificial Sequence
  synthetic peptide
  misc_feature (1)..(1): V or Aib
  misc_feature (4)..(4): W, F, or L
  misc_feature (7)..(7): A, L, F, or Aib
  misc_feature (8)..(8): L or F
  misc_feature (10)..(10): A, Aib or N
  misc_feature (11)..(11): L or F
  misc_feature (12)..(12): A, L, F, or Aib
  misc_feature (15)..(15): L or F
  misc_feature (16)..(16): A or Aib
  misc_feature (18)..(18): L or F
  misc_feature (19)..(19): V or Aib
XQKXKEXXQXXXEKXXQXXD

SEQ ID NO: 38 - protein, 20 aa, Artificial sequence
  MOD_STRUCTURE
VQKWKELLQAFLEKLAQFVD

SEQ ID NO: 39 - protein, 20 aa, Artificial Sequence
  Mod_sequence
  MISC_FEATURE (16)..(16): Aib
VQKWKELLQNFLEKLXQFVD

SEQ ID NO: 40 - protein, 20 aa, Artificial Sequence
  synthesized peptide
  MISC_FEATURE (16)..(16): Aib
VQKFKELLQALLEKLXQFVD

SEQ ID NO: 41 - protein, 20 aa, Artificial sequence
  synthesized peptide
  MISC_FEATURE (16)..(16): Aib
VQKFKELLQNLLEKLXQFVD

SEQ ID NO: 42 - protein, 20 aa, Artificial Sequence
  synthesized peptide
  MISC_FEATURE (7)..(7): Aib
  MISC_FEATURE (16)..(16): Aib
VQKFKEXLQNLLEKLXQFVD

SEQ ID NO: 43 - protein, 20 aa, Artificial Sequence
  synthesized peptide
  MISC_FEATURE (7)..(7): Aib
  MISC_FEATURE (16)..(16): Aib
VQKWKEXLQNLLEKLXQFVD

SEQ ID NO: 44 - protein, 20 aa, Artificial Sequence
  synthesized peptide
VQKFKELLQALLEKLAQFVD

SEQ ID NO: 45 - protein, 20 aa, Artificial Sequence
  synthetic peptide
VQKFKELLQNLLEKLAQFVD

SEQ ID NO: 46 - protein, 20 aa, Artificial Sequence
  synthetic peptide
  MISC_FEATURE (16)..(16): Aib
VQKWKELLQAFLEKLXQFVD

SEQ ID NO: 47 - protein, 20 aa, Artificial Sequence
  synthetic peptide
  misc_feature (16)..(16): Xaa can be any naturally occurring amino acid
VQKWKELLQNFLEKLXQFVD

SEQ ID NO: 48 - protein, 20 aa, Artificial Sequence
  synthetic peptide
  misc_feature (16)..(16): Xaa can be any naturally occurring amino acid
VQKFKELLQNFLEKLXQFVD

SEQ ID NO: 49 - protein, 20 aa, Artificial Sequence
  synthetic peptide
  misc_feature (7)..(7): Xaa can be any naturally occurring amino acid
VQKWKEXLQAFLEKLAQFVD

SEQ ID NO: 50 - protein, 20 aa, Artificial Sequence
  synthetic peptide
  MISC_FEATURE (7)..(7): Aib
VQKWKEXLQNFLEKLAQFVD

SEQ ID NO: 51 - protein, 20 aa, Artificial Sequence
  synthetic peptide
  MISC_FEATURE (7)..(7): Aib
VQKFKEXLQNFLEKLAQFVD

SEQ ID NO: 52 - protein, 20 aa, Artificial Sequence
  synthetic peptide
VQKWKEFLQAFAEKFAQFVD

SEQ ID NO: 53 - protein, 20 aa, Artificial Sequence
  synthetic peptide
  MISC_FEATURE (12)..(12): Aib
VQKWKEFLQAFXEKFAQFVD

SEQ ID NO: 54 - protein, 20 aa, artificial sequence
  synthetic peptide
  MISC_FEATURE (12)..(12): Aib
  MISC_FEATURE (16)..(16): Aib
VQKWKEFLQAFXEKFXQFVD

SEQ ID NO: 55 - protein, 20 aa, artificial sequence
  synthetic peptide
  MISC_FEATURE (10)..(10): Aib
VQKWKEFLQXFAEKFAQFVD

SEQ ID NO: 56 - protein, 20 aa, Artificial Sequence
  synthetic peptide
  MISC_FEATURE (10)..(10): Aib
  misc_feature (12)..(12): Xaa can be any naturally occurring amino acid
VQKWKEFLQXFXEKFAQFVD

SEQ ID NO: 57 - protein, 20 aa, Artificial Sequence
  synthetic peptide
  MISC_FEATURE (10)..(10): Aib
  MISC_FEATURE (12)..(12): Aib
  MISC_FEATURE (16)..(16): Aib
VQKWKEFLQXFXEKFXQFVD

SEQ ID NO: 58 - protein, 20 aa, Artificial Sequence
  synthetic peptide
VQKWKELLQNFLEKLAQFVD

SEQ ID NO: 59 - protein, 20 aa, artificial sequence
  Synthetic peptide
  MISC_FEATURE (1)..(1): V, I, Aib
  MISC_FEATURE (2)..(2): Q, N
  MISC_FEATURE (3)..(3): K, R, H, Orn
  MISC_FEATURE (4)..(4): W, F, Y, I, V, L
  MISC_FEATURE (5)..(5): K, R, H, Orn
  MISC_FEATURE (6)..(6): E, D
  MISC_FEATURE (7)..(7): A, G, S, L, F, V, Aib, Amv
  MISC_FEATURE (8)..(8): L, I, V, W, Aib, Amv, F
  MISC_FEATURE (9)..(9): Q, N
  MISC_FEATURE (10)..(10): A, G, S, Aib, Amv, V, N
  MISC_FEATURE (11)..(11): L, I, V, W, Aib, Amv, F
  MISC_FEATURE (12)..(12): A, G, S, L, F, V, Amv, Aib
  MISC_FEATURE (13)..(13): E, D
  MISC_FEATURE (14)..(14): A, G, S, V, Aib, Amv
  MISC_FEATURE (15)..(15): L,I, V, W, Aib, Amv, F
  MISC_FEATURE (16)..(16): Q, N
  MISC_FEATURE (17)..(17): Q, N
  MISC_FEATURE (18)..(18): L, I, V, W, Aib, Amv, F
  MISC_FEATURE (19)..(19): V, I, Aib
  MISC_FEATURE (20)..(20): E, D
XXXXXXXXXXXXXXXXXXXX

SEQ ID NO: 60 - protein, 20 aa, Artificial Sequence
  synthetic peptide
  MISC_FEATURE (16)..(16): Aib
VQKLKELLQNLLEKLXQFVD

SEQ ID NO: 61 - protein, 20 aa, Artificial Sequence
  synthetic peptide
  MISC_FEATURE (14)..(14): Aib
VQKWKELLQNFLEXLKQFVD

SEQ ID NO: 62 - protein, 20 aa, Artificial Sequence
  synthetic peptide
VQKWKELLQNFLEVLKQFVD

SEQ ID NO: 63 - protein, 20 aa, Artificial Sequence
  synthetic peptide
  misc_feature (18)..(18): Xaa can be any naturally occurring amino acid
VQKWKELLQNFLEFLKQXVD

SEQ ID NO: 64 - protein, 20 aa, Artificial Sequence
  synthetic peptide
  MISC_FEATURE (14)..(14): Aib
VQKFKELLQNFLEXLKQFVD

SEQ ID NO: 65 - protein, 20 aa, Artificial Sequence
  synthetic peptide
VQKFKELLQNFLEVLKQFVD

SEQ ID NO: 66 - protein, 20 aa, Artificial Sequence
  synthetic proteing
  MISC_FEATURE (18)..(18): Aib
VQKFKELLQNFLEFLKQXVD

SEQ ID NO: 67 - protein, 20 aa, Artificial Sequence
  synthetic peptide
  MISC_FEATURE (14)..(14): Aib
VQKWKELLQNFLEXFKQLVD

SEQ ID NO: 68 - protein, 20 aa, Artificial Sequence
  synthetic peptide
  MISC_FEATURE (18)..(18): Aib
VQKWKELLQNFLELFKQXVD

SEQ ID NO: 69 - protein, 20 aa, artificial sequence
  synthetic protein
  MISC_FEATURE (12)..(12): Aib
VQKWKELLQNFXELLKQFVD

SEQ ID NO: 70 - protein, 20 aa, Artificial Sequence
  synthetic peptide
  MISC_FEATURE (8)..(8): Aib
  MISC_FEATURE (14)..(14): Aib
VQKWKELXQNFLEXLKQFVD

SEQ ID NO: 71 - protein, 20 aa, Artificial Sequence
  synthetic peptide
  MISC_FEATURE (7)..(7): Aib
  MISC_FEATURE (14)..(14): Aib
VQKWKEXLQNFLEXLKQFVD

SEQ ID NO: 72 - protein, 20 aa, Artificial Sequence
  synthesized peptide
  MISC_FEATURE (7)..(7): Aib
  MISC_FEATURE (14)..(14): Aib
  misc_feature (19)..(19): Xaa can be any naturally occurring amino acid
VQKWKEXLQNFLEXLKQFXD